(19) United States
(12) Patent Application Publication
(10) Pub. No.: US 2022/0387750 A1
(43) Pub. Date: Dec. 8, 2022

(54) FOLEY CATHETER AND CORRESPONDING TRAY PACKAGING SYSTEM

(71) Applicant: Medline Industries, LP, Northfield, IL (US)

(72) Inventor: Eric Hendrickson, Arlington Heights, IL (US)

(21) Appl. No.: 17/372,149

(22) Filed: Jul. 9, 2021

Related U.S. Application Data
(60) Provisional application No. 63/208,461, filed on Jun. 8, 2021.

(51) Int. Cl. A61M 25/00

(52) U.S. Cl. CPC A61M 25/002; A61M 25/0017; A61M 2205/0222; A61B 10/007

(57) ABSTRACT

A medical procedure kit includes a first tray. A second tray situates atop the first tray. The second tray includes preformed compartments configured to receive and house a plurality of medical implements. A Foley catheter positioned within a Foley catheter compartment of the plurality of preformed compartments of the second tray. A drainage system stowed within an interior of the first tray. A third tray can be situated atop the second tray. The second tray can include a plurality of catheter retention devices. A planar substrate can be positioned beneath the first tray. A Foley catheter compartment can define a barrier wall extending across a majority of its length. A cutout in the second tray and another cutout in the first tray can be positioned at opposite ends of the kit. These features can be used alone or in combination, and many can even be applied to a single-layer tray.

### CROSS REFERENCE TO PRIOR APPLICATIONS

This application claims priority and benefit under 35 U.S.C. §119(e) from U.S. Provisional Application No. 63/208,461, filed Jun. 8, 2021, which is incorporated by reference for all purposes.

### BACKGROUND

### Technical Field

This disclosure relates generally to Foley catheters, and more particularly to Foley catheter packaging systems.

### Background Art

Medical devices, including surgical instruments, supplies, and so forth, are generally shipped from manufacturer to medical services provider in sterile packaging. For example, a scalpel may be shipped to a surgeon in a plastic, vacuum-sealed, sterile package. Similarly, bandages may be shipped in paper, plastic, or paper composite sterile wrappers. When the medical services provider is ready to use the medical supply, the sterile package is removed. The medical services provider then uses the object in accordance with the procedure being performed.

While conventional packaging works well for objects having a generally unchanging form factor, special considerations have to be taken into consideration for some medical supplies. By way of example, Foley catheters and their corresponding tubing and other flexible equipment is generally shipped in a coiled configuration. Once the sterile packaging is removed, the tubing and other coiled materials must be uncoiled prior to use. Care must be taken in shipping, unwrapping, and using coiled assemblies. For instance, if a Foley catheter is inadvertently bent, kinked, or otherwise damaged, it may no longer be suitable for use. It would be advantageous to have an improved packaging system for Foley catheters.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates a top, front, right perspective view of one explanatory tray.

FIG. 2 illustrates a top, front, left perspective view of one explanatory tray

FIG. 3 illustrates a top plan view of explanatory tray.

FIG. 4 illustrates a front elevation view of one explanatory tray.

FIG. 5 illustrates a cut-away, left elevation view of one explanatory tray.

FIG. 6 illustrates a bottom plan view of one explanatory tray.

FIG. 7 illustrates a top plan view one explanatory Foley catheter, coiled tubing, and fluid drainage bag.

FIG. 8 illustrates a top plan view a medical kit.

FIG. 9 illustrates a perspective view of another medical kit.

FIG. 10 illustrates a top plan view of still another medical kit.

FIG. 11 illustrates a perspective view of another medical kit.

FIG. 12 illustrates a top plan view of one explanatory medical kit in accordance with one or more embodiments of the disclosure.

FIG. 13 illustrates a bottom plan view of a tray of a medical kit.

FIG. 14 illustrates an elevation view of a medical kit.

FIG. 15 illustrates disassembled components of one explanatory medical kit in accordance with one or more embodiments of the disclosure.

FIG. 16 illustrates assembled components of one explanatory medical kit in accordance with one or more embodiments of the disclosure.

FIG. 17 illustrates an elevation view of one explanatory medical kit in accordance with one or more embodiments of the disclosure.

FIG. 18 illustrates a top plan view of disassembled components of another explanatory medical kit in accordance with one or more embodiments of the disclosure.

FIG. 19 illustrates assembled components of one explanatory medical kit in accordance with one or more embodiments of the disclosure.

FIG. 20 illustrates an elevational view of one explanatory medical kit in accordance with one or more embodiments of the disclosure.

FIG. 21 illustrates a top plan view of still another explanatory medical kit in accordance with one or more embodiments of the disclosure.

FIG. 22 illustrates an elevational view of one explanatory medical kit in accordance with one or more embodiments of the disclosure.

FIG. 23 illustrates a perspective view of still another explanatory medical kit in accordance with one or more embodiments of the disclosure.

FIG. 24 illustrates another elevation view of the medical kit of FIG. 23.

FIG. 25 illustrates a perspective view of still another explanatory medical kit in accordance with one or more embodiments of the disclosure.

FIG. 26 illustrates various embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DISCLOSURE

Before describing in detail embodiments that are in accordance with the present disclosure, it should be observed that the embodiments reside primarily in combinations of method steps and apparatus components related to packaging Foley catheters, their corresponding coiled tubing, fluid drain bags, and other components in a tray. Alternate implementations are included, and it will be clear that any method steps may be executed out of order from that shown or discussed, including substantially concurrently or in reverse order, depending on the desired application. Accordingly, the apparatus components and method steps have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, it is expected that one of ordinary skill, notwithstanding possibly significant effort and many design choices motivated by, for example, available time, current technology, and economic considerations, when guided by the concepts and principles disclosed herein will be readily capable of generating such assemblies and executing such method steps with minimal experimentation.

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions.

As used herein, components may be “operatively coupled” when information can be sent between such components, even though there may be one or more intermediate or intervening components between, or along the connection path. The terms “substantially” and “about” are used to refer to dimensions, orientations, or alignments inclusive of manufacturing tolerances. Thus, a “substantially orthogonal” angle with a manufacturing tolerance of plus or minus two degrees would include all angles between 88 and 92, inclusive. Also, reference designators shown herein in parenthesis indicate components shown in a figure other than the one in discussion. For example, talking about a device (10) while discussing figure A would refer to an element, 10, shown in figure other than figure A.

Embodiments of the disclosure provide a medical procedure kit comprising a tray. In one or more embodiments, the tray comprises a first compartment and a second compartment. The first compartment is at least partially bounded by a first base member in one or more embodiments. The second compartment is at least partially bounded by a second base member in one or more embodiments.

In one or more embodiments, at least one syringe is situated within the first compartment. In one or more embodiments, a Foley catheter is situated in the second compartment. In one or more embodiments, the Foley catheter is operatively coupled to coiled tubing. In one or more embodiments, the coiled tubing is operatively coupled to a fluid drain bag. In one or more embodiments where the Foley catheter is disposed in the second compartment, the operatively coupled coiled tubing and fluid drain bag are also disposed within the second compartment. In other embodiments, such as when a medical kit is configured as a two-layer tray, the Foley catheter can be placed in the top tray with at least some of the coiled tubing, while the remainder of the coiled tubing and the fluid drain bag can be placed in the lower tray.

Embodiments of the present disclosure further provide a medical procedure kit that includes medical products for performing a medical procedure. In one embodiment, the medical procedure kit is configured for a Foley catheterization procedure. Such an embodiment will be used herein for illustration purposes. However, it will be clear to those of ordinary skill in the art having the benefit of this disclosure that embodiments of the disclosure are not so limited. Other medical procedure kits for performing other procedures could be substituted for the illustrative Foley catheterization procedure kit disclosed herein by substituting other medical implements for the Foley catheterization implements.

When a Foley catheter assembly is inserted into a patient, sterile water may be used to inflate the balloon of the Foley catheter. Additionally, the Foley catheter may be coated in a lubricating jelly prior to insertion into the patient. Fluids and other samples may then be monitored and obtained from the patient as they pass into the Foley catheter, through the coiled tubing (which may be uncoiled at this point) and into a syringe via a Luer access port or into the fluid drain bag. Embodiments of the present disclosure provide a tray configured to accommodate not only the Foley catheter, the operatively coupled coiled tubing, and the operatively coupled fluid bag, but also syringes containing fluids such as sterile water or aqueous lubricants. Further, the tray can accommodate a sterile specimen jar for capturing samples taken from the patient as they pass into the Foley catheter, through the coiled tubing (which may be uncoiled at this point) and into a syringe via a Luer access port or into the fluid drain bag.

In addition to simply accommodating these corresponding medical devices, in one or more embodiments the tray is configured to provide the medical services provider with mnemonic devices instructing them in which order to use each device. For example, a compartment containing syringes, in one embodiment, includes a base member that presents each syringe at, for example, an easy to reach angle and/or at different heights based upon order in which each syringe is used in a Foley catheterization procedure.

Another advantage of embodiments of the present disclosure is that compartments have multi-purpose functionality. For example, in one embodiment, a compartment configured to accommodate a syringe having lubricating jelly disposed therein is also configured for use as a lubricating jelly applicator or lubricating jelly application compartment. In one or more embodiments, a medical services provider first dispenses the lubricating jelly into the compartment from which the syringe was taken. The medical services provider then passes at least a tip of the Foley catheter from the second compartment into the first compartment and into the lubricating jelly. Advantageously, the tray not only serves as a shipping and storage container for an assembly of devices used with a Foley catheter procedure, but also as an application device to assist a medical services provider in using those products together, and without compromising the sterile field required for proper aseptic technique.

Embodiments of the disclosure provide a medical procedure kit that can include at least one tray. The kit can include only a single tray in some embodiments. In other embodiments, the kit includes multiple trays stacked a two-layered configuration. Trays configured in accordance with one or more embodiments of the disclosure can include some clamps and/or clips to better hold the Foley catheter within a tray. In one or more embodiments, the clamps and/or clips define small arches through which the Foley catheter may pass. The use of clamps and/or clips is advantageous in two-layer kits, as many health care services providers will initially lift the top tray off the bottom tray when accessing the medical implements situated within the tray. When they do, and when the coiled tubing attached to the Foley catheter is placed in the bottom tray, the Foley catheter may be pulled out of the upper tray if not for the clamps and/or clips. Advantageously, the clips and/or clamps help hold the Foley catheter in place as the upper tray is removed from the lower tray with the Foley catheter situated in the upper tray.

In other two-layer stacked tray implementations, embodiments of the disclosure contemplate that due to the manufacturing process used to manufacture the upper tray with its many contours and compartments, the bottom of this upper tray will have a lot of protrusions. When the coiled tubing attached to the Foley catheter is placed in the bottom tray, these contours and protrusions can cause the coiled tubing to snag when the bottom tray is moved relative to the upper tray. Advantageously, embodiments of the disclosure provide a solution to this problem by providing a panel that is placed beneath the upper tray. The inclusion of this panel prevents snagging.

In still other embodiments, what appears to be a conventional two-layer kit actually includes the upper tray of the two-layer tray assembly configured as multiple trays dividing the upper tray in two. In one or more embodiments, the povidone-iodine (PVP) and syringes are situated in one upper tray, while the Foley catheter is in a second upper tray. A bottom tray supports this two-component upper tray assembly. This use of two components advantageously makes it easier to reach the medical implements situated in the bottom tray out of the bottom tray without creating issues with the Foley catheter situated in the upper tray assembly.

In one or more embodiments in a two-layer tray assembly, embodiments of the disclosure provide a bottom tray being manufactured from plastic instead of cardboard for additional rigidity.

In still other embodiments, a three-layer medical kit is provided. A third tray houses drapes and/or gloves in a separate tray so that a medical practitioner need not reach into the sterile field to get them. In other two-layer systems, the upper tray slides out from the lower tray.

In contrast to prior art two-layer tray systems having a lower tray with an open end and a closed end, with the two trays arranged such that the closed end bounds an opening for coiled tubing coupled to a Foley catheter in the upper tray, which requires a health care services provider to lift the top tray and rotate the same 180-degrees when positioning the top tray towards the patient (i.e., to the left of the bottom train if patient is to the left), embodiments of the disclosure advantageously position the top tray rotated from the start, thereby reducing the chances of a spill occurring.

In still other embodiments, a two-layer system includes an upper tray that slides, optionally on rails, along the top of the bottom tray. Such an embodiment could also include a channel connection so that the top tray slides out from the bottom tray. Where a Foley catheter is situated in the upper tray, the rail and/or channel-based system makes the Foley catheter less likely to move when separating the upper and lower tray.

Other embodiments will be described below. Variants will be obvious to those of ordinary skill in the art having the benefit of this disclosure. Illustrating by example, the swab sticks may be saturated with chlorhexidine gluconate (CHG) rather than PVP. Additionally, other catheters could be substituted for the Foley catheter in the various embodiments, including a 3-way Foley catheter, a Coude tip with temp sensing Foley catheter, or other type of catheter. Still other embodiments will be obvious to those of ordinary skill in the art having the benefit of this disclosure.

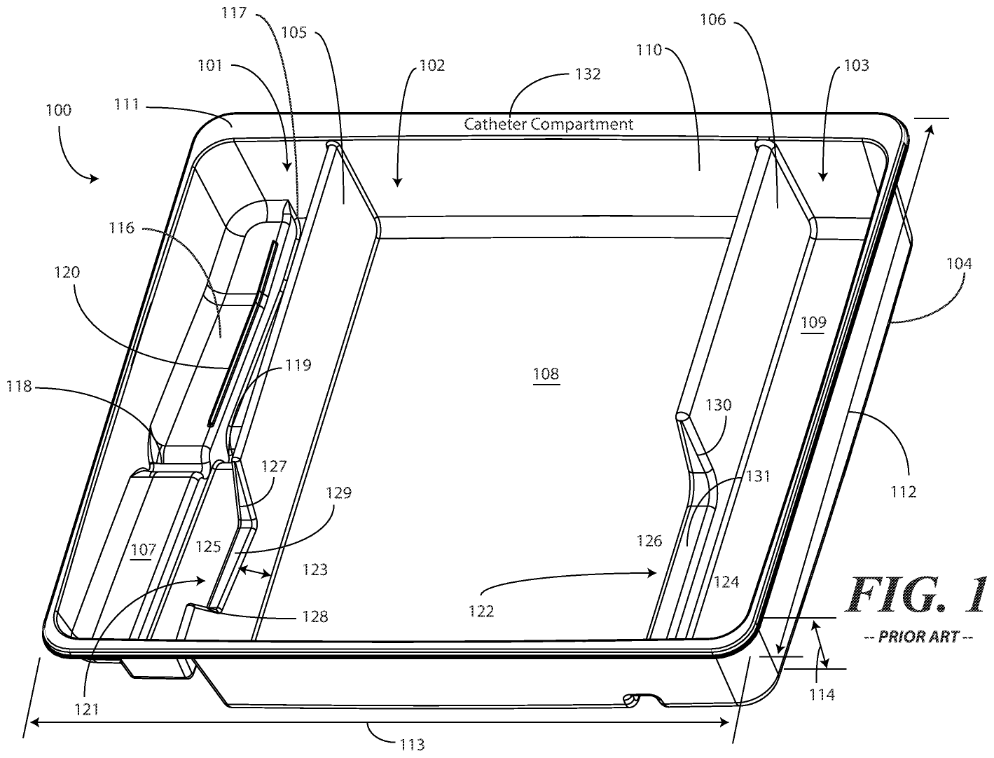

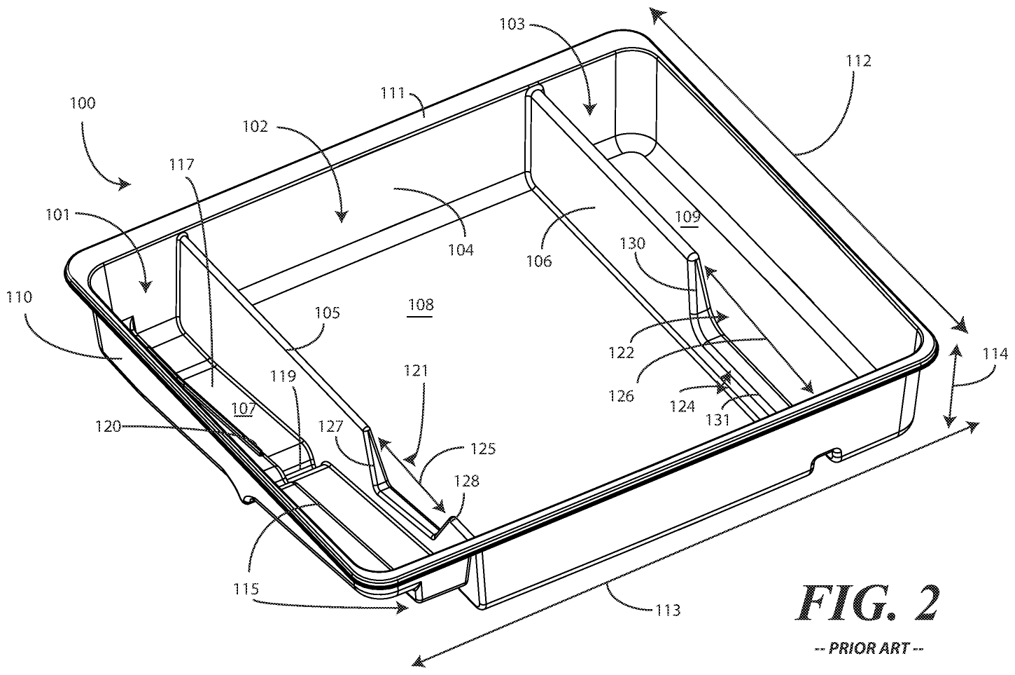

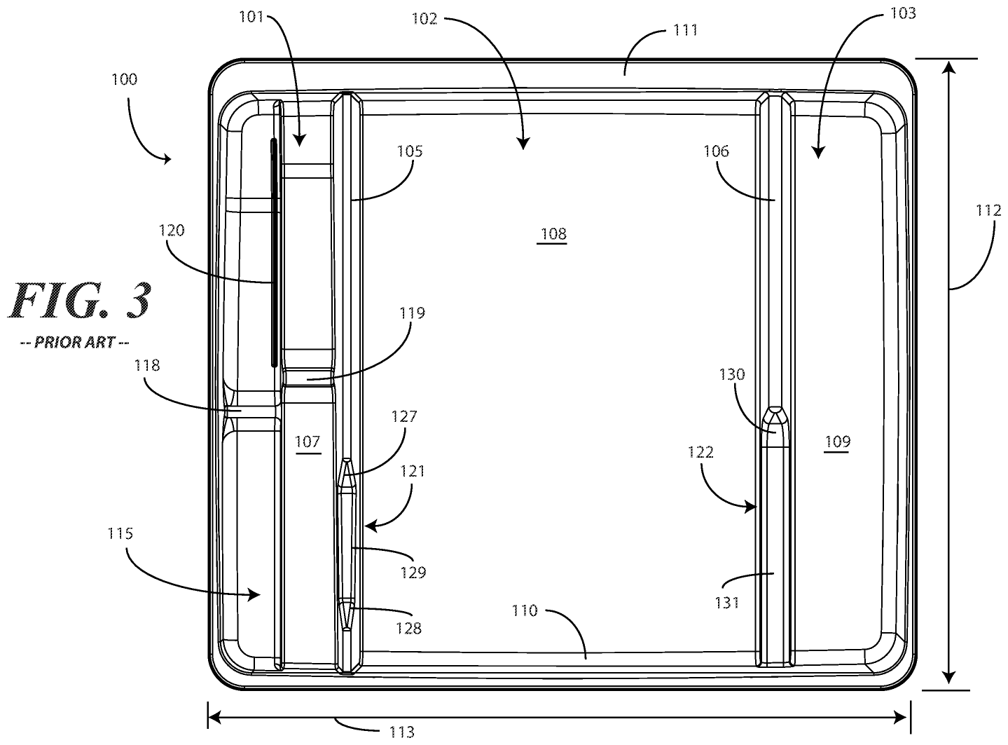

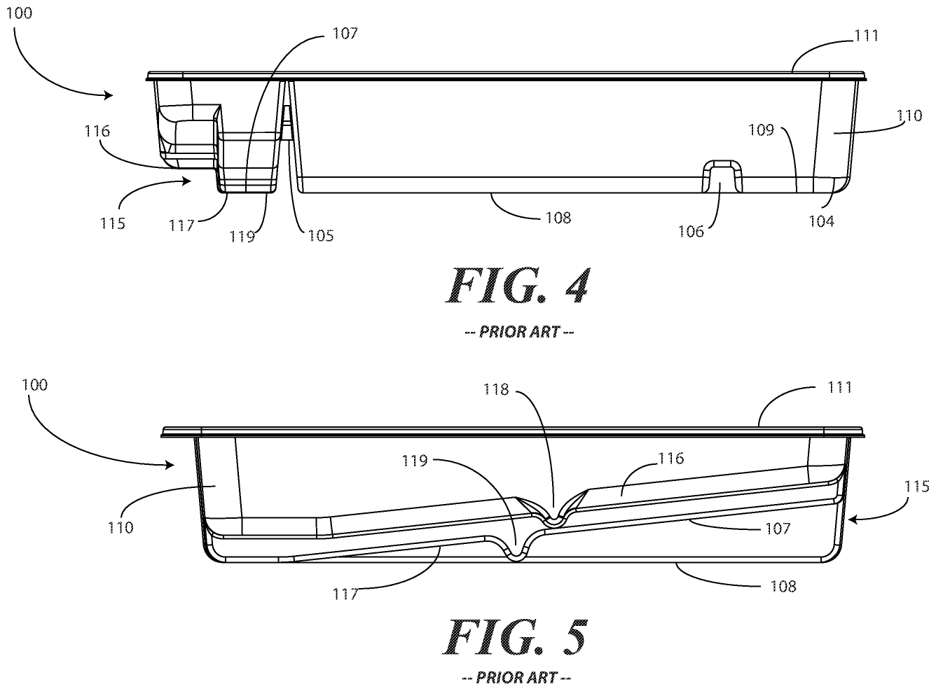

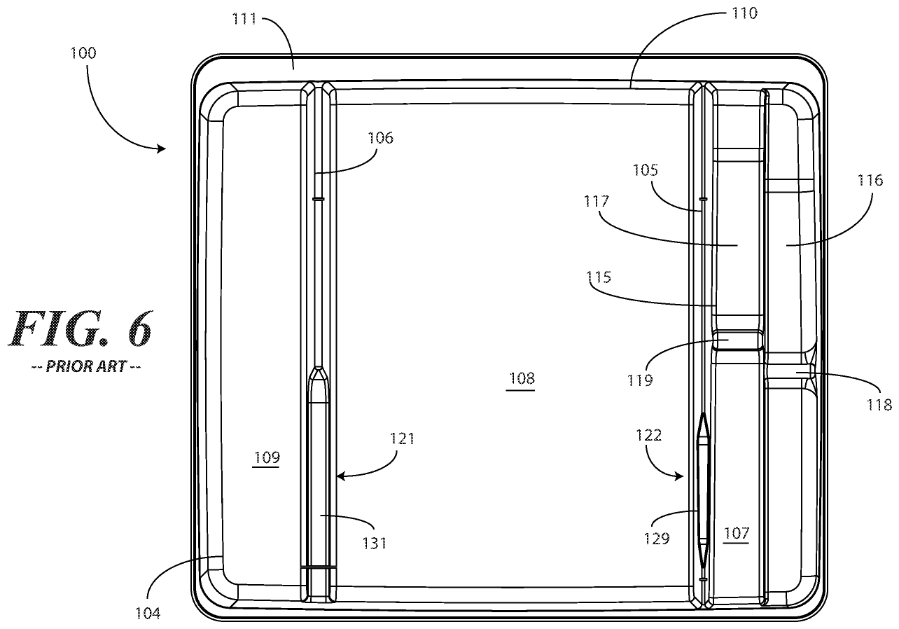

Turning now to FIGS. 1-6, illustrated therein are views of one embodiment of a tray 100 configured to accommodate a Foley catheter, its operatively coupled coiled tubing, and its operatively coupled fluid drain bag. FIG. 1 illustrates a top, front right perspective view of the tray 100. FIG. 2 illustrates a top, front, left perspective view of the tray 100. FIG. 3 illustrates a top plan view of the tray 100. FIG. 4 illustrates a front elevation view of the tray 100. FIG. 5 illustrates a cut-away, left elevation view of the tray 100. Likewise, FIG. 6 illustrates a bottom plan view of the tray 100. For simplicity of discussion, these figures will be referred to collectively with like reference numerals referring to identical or functionally similar elements throughout the separate views.

The tray 100 is formed by a contoured surface 104 that defines the various features and compartments of the tray 100. The contoured surface 104 of the tray 100 can be manufactured in various ways. For example, the tray 100 can be thermally formed on a mold from a soft thermoplastic, such as styrene or polystyrene. The tray 100 can be injection molded. The tray can be poured on a mold using a quick setting plastic, epoxy, or resin.

Explanatory dimensions of the tray 100 are as follows: The length 112 can be between nine and twelve inches, such as ten inches. One illustrative length 112 may be 10.380 inches. Similarly, the width 113 can be between eight and eleven inches, such as nine inches. One illustrative width 113 is 9.250 inches. The height 114 can be between one and three inches. One illustrative height 114 is 1.750 inches.

The tray 100 includes three main compartments: a first compartment 101, a second compartment 102, and a third compartment 103. The first compartment 101 is separated from the second compartment 102 by a first barrier 105. The second compartment 102 is separated from the third compartment 103 by a second barrier 106.

The compartments are open from the top of the tray 100—the top being opposite the base members of the tray 100—and are bounded on the bottom by a first compartment base member 107, a second compartment base member 108, and a third compartment base member 109. The compartments are bounded on the sides by a perimeter wall 110. The perimeter wall 110 ends in a horizontal flange 111 extending substantially orthogonally from the perimeter wall 110. The top of the tray 100 could have a hinged or snap-coupled lid that is opened or removed to reveal the compartments there beneath. Similarly, the horizontal flange 111 can be omitted, with each of the first compartment 101, the second compartment 102, and the third compartment 103 being bounded by a wall, be it the perimeter wall 110 or another wall, that extends distally from the various compartment base members 107,108,109 to a terminating edge.

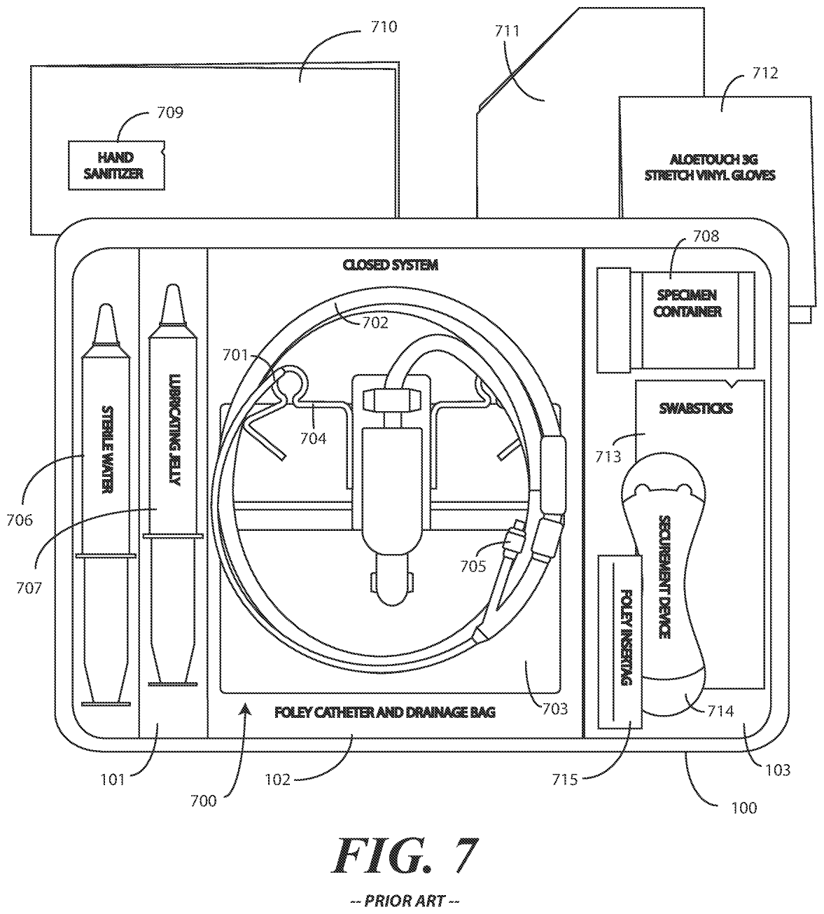

The tray 100 is configured to hold or otherwise accommodate the necessary devices and materials to perform a Foley catheter-based procedure on a patient. The tray 100 is configured to hold not only the Foley catheter, its operatively coupled coiled tubing, and its operatively coupled fluid drain bag, but the medical devices corresponding to Foley catheter use as well. Using one illustrative procedure as an example, the following devices will be used: a syringe holding sterile water, a syringe holding lubricating jelly or another equivalent lubricant, a catheter assembly, skin cleansing or preparation materials, and a specimen jar. The various compartments and features of the tray 100 shown in FIGS. 1-6 will be described for use with these devices. As will be described in more detail below with reference to FIG. 7, additional objects can be included with the tray, such as one or more towels, a drape to cover the patient, rubber gloves, hand sanitizing materials, swab sticks, a securement device, a Foley insert tag, a printed instruction pamphlet, and so forth.

For procedures using the syringe holding sterile water, syringe holding lubricating jelly, the Foley catheter, its operatively coupled coiled tubing, and its operatively coupled fluid drain bag, and specimen jar, the tray 100 is configured such that these objects are ordered in accordance with their order of use during the Foley catheterization procedure. For example, the tray 100 includes a first compartment 101 for accommodating one or more syringes, a second compartment 102 for accommodating the Foley catheter, its operatively coupled coiled tubing, and its operatively coupled fluid drain bag, and optionally one or more syringes, and a third compartment 103 for accommodating at least the specimen jar. These devices stowed in the various compartments will be illustrated and described with respect to FIG. 7 below. The discussion of FIGS. 1-6 will include the features of the tray 100 that make the tray 100 suitable for accommodating these devices.

The first compartment base member 107 is contoured. The first compartment base member 107 can include features that hold any syringes stored within the first compartment 101 at different heights. The first compartment base member 107 includes a stair-stepped contour 115 suitable that supports syringes at different heights.

A first step portion 116 of the stair-stepped contour 115 may be at a different height within the tray 100 than a second step portion 117 of the stair-stepped contour 115. The first step portion 116—which is disposed farther from the first barrier 105 than the second step portion 117—is shallower than the second step portion 117. Said differently, the second step portion 117 is disposed at a greater depth within the tray 100 than the first step portion 116.

The contours of the first compartment 101 can serve as mnemonic device when for medical implements stored within the first compartment 101. For example, it may be intuitive that a higher syringe may need to be used first. Accordingly, placing this “first to be used” syringe on a higher step portion will indicate that this syringe should to be used first. This intuition is further enforced when the higher position is situated farther to the left in a left-to-right usage, i.e., like reading a book, configuration. Thus, a user receives a mnemonic reminder to use a syringe disposed on the first step portion 116 prior to a syringe disposed on the second step portion 117, as it is both higher and farther to the left.

Where syringes are stowed in the first compartment 101, the first compartment base member 107 can further be configured for syringe ease of use. For example, the first compartment base member 107 is inclined relative to other compartment base members. In FIGS. 1-6, the second compartment base member 108 and third compartment base member 109 are substantially coplanar with each other. Further, the second compartment base member 108 and third compartment base member 109 are generally flat in these views. The first compartment base member 107 could be substantially flat.

In this illustrative tray 100, the first compartment base member 107 is configured to be inclined relative to one or both of the second compartment base member 108 and third compartment base member 109. As such, the stair-stepped contour 115 forms a ramp upon which syringes may be placed so that the plunger of each syringe is predisposed to project upward and out of the tray 100. Said differently, the stair-stepped contour 115 is configured such that the first step portion 116 and the second step portion 117 are disposed in a non-parallel orientation relative to the second compartment base member 108. This configuration makes it easier for a medical services provider to grasp the syringes and remove them from the tray 100.

The first compartment base member 107 may include other features suitable for accommodating one or more syringes as well. Any of the first compartment base member 107, the first step portion 116, or the second step portion 117 can include recesses 118,119 for accommodating a syringe flange. These recesses 118,119 generally function to prevent the syringes from sliding lengthwise within the first compartment 101. Similarly, any of the first compartment base member 107, the first compartment sidewalls, the first step portion 116, or the second step portion 117 include protrusions 120 that help to prevent the syringes from sliding laterally within the first compartment 101.

One or both of the first barrier 105 and the second barrier 106 include optional openings disposed therein. In FIGS. 1-6, the first barrier 105 includes a first opening 121 between the first compartment 101 and the second compartment 102. Similarly, the second barrier 106 includes a second opening 122 between the second compartment 102 and the third compartment 103. Each of these openings has an opening depth associated therewith. Similarly, each opening has an opening width associated therewith. In FIGS. 1-6, the first opening 121 is bounded by a first opening base member 129 and two inclined first opening side members 127,128, while the second opening 122 is bounded by a second opening base member 131, an inclined second opening side member 130, and the perimeter wall 110.

While the opening depths can be the same, the opening depths are different in FIGS. 1-6. For example, in FIGS. 1-6, the first opening 121 has a first opening depth 123 that is less than the second opening depth 124 of the second opening 122. Similarly, the opening widths are different. For example, in FIGS. 1-6, the first opening 121 has a first opening width 125 that is less than the second opening width 126 of the second opening 122. Such a disparity in opening depths and widths, as well as the inclusion of inclined opening side members, provides an advantage in some applications.

For instance, in many Foley catheter procedures a pair of syringes—such as syringes having a one-half inch diameter—fits easily into the first compartment 101 when the tray 100 is made with the illustrative dimensions set forth above. However, some procedures require one or more of the syringes to be larger. Additionally, in still other procedures syringes are used in different orders. Where syringes that are larger in diameter are required, these larger syringes are capable of nesting within the first opening 121 and second opening 122. The inclined opening side members prevent the syringe from moving lengthwise, while the disparate opening heights present the plunger of the syringe to the medical services provider for easy removal from the tray 100. Where the syringes are used in different orders, or simply for design choice, one syringe can be placed in the first compartment 101, while another syringe can be placed in the second compartment 102, and so forth.

The stair-stepped contour 115, working in tandem with the first opening 121, gives the tray additional advantages. For instance, the first compartment base member 107 allows the first compartment 101 to be used as a lubricant applicator for the catheter.

Specifically, the medical services provider may dispense the lubricating jelly into the first compartment 101 after removing the syringes therefrom. This lubricating jelly is dispensed along the second step portion 117. Where included, and where lower than the first step portion 116, the second step portion 117 serves as a channel in which the lubricating jelly may spread. A medical services provider may then pass the at least a tip of the Foley catheter from the first compartment 101 into the second compartment 102, optionally through the first opening 121, and into the lubricating jelly. The tip or more of the Foley catheter passes through the channel formed by the second step portion 117, i.e., along the second step portion 117 through the dispensed lubricating jelly. From there, the Foley catheter is passed out the top of the tray 100 to the patient.

This feature of the tray 100 greatly eases the application of lubricating jelly to the catheter. The tray 100 is packaged with printed instructions showing the medical services provider how to apply lubricating jelly in this manner.

Alternative methods may be used to apply the lubricating jelly as well. For example, the lubricating jelly can be dispensed directly onto the Foley catheter and/or its operatively coupled tubing while these components are situated in or above either the first compartment 101 or the second compartment 102. Excess lubricant falling from the catheter tubing can then collect, and be retained, in any of the first compartment 101, the second step portion 117 of the first compartment 101, or the second compartment 102.

This particular feature highlights another advantage of the “compartmentalized” structure. As the tray 100 includes multiple compartments, various tasks associated with a Foley catheterization procedure can be completed while keeping the Foley catheter within the tray 100. The ability to keep the catheter in the tray 100 reduces the risk that the Foley catheter, its operatively coupled coiled tubing, and its operatively coupled fluid drain bag, or corresponding devices, will be contaminated with bacteria or microbes on other objects within the procedure room.

Illustrating by example, when the first compartment 101 is used to apply lubricating jelly to the Foley catheter or its operatively coupled coiled tubing, the lubricating jelly can be applied while these components are completely contained within the confines of the tray 100. This reduces the risk that the Foley catheter or its operatively coupled coiled tubing will become contaminated. This correspondingly reduces the risk of infection for the patient receiving the catheter.

Systems in which the catheterization procedure components are shipped in separate and/or stacked containers may contribute to substandard techniques in that the catheter can become contaminated when moving it from its shipping container. Consequently, the patient can be at an elevated risk of infection as the catheter is moved from one tray to another. A solution to this problem is the provision of a tray 100 with compartments. Further, the first compartment 101 allows the Foley catheter can stay in place during and after lubrication. By having easy access to the components disposed in the tray 100, the medical services provider can more easily prepare and use the components within the tray 100. This helps to minimize the risk of contaminating the patient or the sterile field during the procedure.

The second step portion 117 is configured to be inclined at a shallower angle than the first step portion 116 in at least a portion opposite the recess 119 from the first opening 121. When configured in such a fashion, the second step portion 117 includes a “cutdown” so that the catheter can stay within the channel both during and after lubrication.

Additionally, the Foley catheter can be placed in both the first opening 121 and second opening 122 during lubrication. When positioned in this configuration, the second opening 122 helps to align the catheter with the first opening for easy passage through the lubrication channel formed by the second step portion 117.

The tray 100 of FIGS. 1-6 includes additional advantages. For example, instructions 132 or other graphical indicia can be printed, placed upon, or molded into the tray. The graphical indicia are molded into the horizontal flange 111. However, it can be molded into other portions of the tray 100, including the base members of the compartments, along the barriers, and so forth. Compartment designations can be placed above each compartment to ensure the medical services provider uses the correct device or material at the correct time. Expiratory dates for materials or devices disposed within the tray 100 may be molded into portions of the tray 100.

Another advantage of the tray 100 is that its compartmentalized configuration helps to reduce the risk of contaminating a patient or compromising the sterile nature of the components stored in the tray 100. Since a Foley catheter, its operatively coupled coiled tubing, and its operatively coupled fluid drain bag, and medical devices corresponding to Foley catheter use, are stored within the same tray 100, the risk of cross-contamination between sterile work areas and non-sterile spaces is minimized Further, by having the Foley catheter, its operatively coupled coiled tubing, and its operatively coupled fluid drain bag, and the devices corresponding to catheter use stowed in a one-level tray rather than a multi-level, stacked configuration, the medical services provider can more easily prepare and use the catheter and corresponding devices disposed within the tray 100.

Turning now to FIG. 7, illustrated therein is one explanatory embodiment of a medical kit. A catheter assembly comprises a Foley catheter 701, its operatively coupled coiled tubing 702, and its operatively coupled fluid drain bag 703. As shown in FIG. 7, the coiled tubing 702 is coupled between the Foley catheter 701 and the fluid drain bag 703.

A clip or other hanger 704 can be coupled to the fluid drain bag 703 so that the fluid drain bag 703 can be coupled to a stand, bedrail, or other object. The coiled tubing 702 may be coupled to the Foley catheter 701, and the fluid drain bag 703 coupled to the coiled tubing 702, before the assembly is placed in the tray 100. Alternatively the Foley catheter 701, the coiled tubing 702, and the fluid drain bag 703 can be separately stored in a tray, with each being coupled together once the tray is opened.

The Foley catheter 701 includes a drainage port 705, which is operatively coupled to the coiled tubing 702. The Foley catheter 701 can optionally include an inflation port. A syringe containing sterile water or other fluid can be coupled to the inflation port. The sterile water or other fluid can be injected into the inflation port so that the balloon of the Foley catheter 701 can be inflated.

The Foley catheter 701 is suitable for insertion into the urethra of a patient. The Foley catheter 701 is an elongated, flexible device. The Foley catheter 701 can be manufactured from flexible material such as polymers, elastomers, flexible steel, or other materials. Other materials suitable for manufacturing the Foley catheter 701 will be obvious to those of ordinary skill in the art.

The Foley catheter 701 includes an insertion end and a second end, which is coupled to the coiled tubing 702. A central lumen passes through the Foley catheter 701 so that fluids may pass from the insertion end to the second end, through the operatively coupled coiled tubing 702, and into the fluid drain bag 703.

When inserted into a patient, an inflatable retaining balloon (not shown) operatively coupled to the Foley catheter 701 can be inflated using the inflation port to retain the Foley catheter 701 within the patient. The balloon can be inflated with sterile water to retain the insertion end of the Foley catheter 701 within the patient's bladder.

FIG. 7 illustrated the tray 100 of FIGS. 1-6 having the catheter assembly 700, syringes 706,707, and a specimen container 708 stored therein as a catheter packaging system. The illustrative catheter packaging system of FIG. 7 includes a tray 100 having a first compartment 101, a second compartment 102, and a third compartment 103.

The first compartment 101 is configured to accommodate syringes 706,707. The second compartment 102 is configured to accommodate a coiled medical device, such as catheter assembly 700. The third compartment 103 is configured to accommodate the specimen container 708. The third compartment 103 can accommodate other materials as well, including skin sanitizers 709 and cleansing liquids, solutions, or gels. Additional devices corresponding to Foley catheter use, including towels 710, drapes 711, rubber gloves 712, one or more swab sticks 713, a Foley catheter securement device 714, a Foley catheter insertion tag 715, and a Foley insertion tag, which is a dated and/or time stamped label that is secured to the catheter tubing once the Foley catheter 701 is inserted, and so forth, can be disposed in the tray 100 as well. As an illustration of this flexibility, one or more towels can be disposed beneath the catheter assembly.

Syringes 706,707 are disposed in the first compartment 101. One syringe 706 contains a sterile liquid, such as sterile water. The other syringe 707 contains a lubricant, such as lubricating jelly. The lubricating jelly can be discharged into the first compartment 101 to lubricate at least a portion of the Foley catheter 701 when the Foley catheter 701 is passed from the second compartment 102 into the first compartment 101.

Once the necessary components are disposed within the tray 100, the tray 100 can be sealed with a wrap to keep the internal components sterile. The wrap can be any of a number of types of material. The wrap can be a central sterile reprocessing (CSR) wrap that is used widely by medical professionals in hospitals, ambulatory surgical centers, and the like during medical procedures. While a CSR wrap is one example of a wrap that can be used, other wraps, such as plastic, cotton, linen, paper, or combinations thereof, can be substituted for the CSR wrap.

The CSR wrap can be folded about the tray 100 for sealing, and can be correspondingly unfolded to reveal the tray 100. Once unfolded, the CSR wrap can then be used in the catheter insertion process. For example, an unfolded CSR wrap can be used to provide a sterile field in which the tray 100 sits for unloading and subsequent use.

Printed instructions can then be attached to, disposed upon, or disposed within the tray 100. The printed instructions can include a health care services portion and a patient portion. The health care services portion can include instructions telling the health care services provider, for example, how to set up a sterile or otherwise clean work environment, how to prepare the catheter assembly 700 disposed within the tray, how to use the other devices within the tray, how to insert the catheter, how to secure the drainage bag to the catheter, how to empty the drainage bag, how to obtain a urine sample, and so forth. The instructions can include pictures or illustrations showing visually how the various steps should be done as well.

The patient portion can include helpful suggestions or instructions for the patient. The patient portion can be detachably coupled to the health care services portion, such as by a perforated line that is easily torn to separate the patient portion from the health care services portion. Examples of suggestions or instructions that may be included in the patient portion include information on what a catheter is, what the patient should understand about the catheter, how to reduce the chance of getting an infection, information about infections commonly associated with catheters, symptoms of infections commonly associated with catheters, and suggestions for home use of the catheter assembly 700. The health care services portion may include an instruction for the health care services provider to detach the patient portion from the health care services portion and instructions to discuss the patient portion with the patient.

The health care services portion can tell the medical services provider how to perform a standard catheterization procedure. For instance, the tray 100 can be equipped with an adhesive label that can be used to identify the patient or specimen in the specimen container 903. Further, a label can be included to mark or otherwise identify the material in the fluid bag attached to the catheter. Such labels can include pre-printed fields, such as date, time and name Further the printed instructions can notify the medical services provider that the devices disposed within the tray 100 are ordered corresponding to use during the catheterization procedure.

The printed instructions can inform the medical services provider of special instructions. For instance, the printed instructions can inform the medical services provider not to leave a catheter in a patient for more than forty-eight hours without a physician's approval. Where the printed instructions include such information, the labels included in the tray 100 may have pre-printed fields for the time of insertion that can be filled in by the medical services provider performing the catheterization procedure.

Once the printed instructions have been affixed to, or placed with, within, or atop the tray 100, the assembly can be sealed in a sterile wrap such as a thermally sealed bag. The thermally sealed bag can optionally include a preformed opening. For example, the opening can include one or more tabs that a health care services provider is instructed to pull to open the bag. Inclusion of a sterile wrap not only keeps the contents within the bag sterile, but also allows the instructions to be included with the tray assembly, yet outside the CSR wrap.

The printed instructions can be disposed atop the CSR wrap such that the health care services portion of the printed instructions is disposed on the top of the printed instructions, with the patient portion being disposed adjacent to the CSR wrap, such as when the printed instructions are configured as an accordion-style folded instruction pamphlet. While the printed instructions of can be configured as a folded, printed, separate article disposed atop the CSR wrap, rather than including separate printed instructions, the instructions for use can be printed on the CSR wrap as well.

Additional instruction materials may be included with the completed assembly as well.

For example, an adhesive instruction tag can be affixed to the sterile wrap. The instruction tag may be adhered to an outer packaging that encloses the tray, the sterile wrap material, or both. For example, the instruction tag can include information regarding whether a catheter procedure is needed. Text such as “Is there a valid clinical reason?” may be included under an instruction to “Stop” that includes the following information:
Before inserting the Foley catheter, at least one of the following conditions should exist:
Acute urinary retention or obstruction
Precise measurement of urinary output needed
Select surgical procedures
Open sacral or perineal wounds in incontinent patient
Prolonged immobilization
End of life care

Further, checklist text may be included, such as “Checklist for Foley Catheter Insertion” included under the word “Check” that includes the following information:
Check Each Box Upon Completion:
Obtain order from physician/provider
Document clinical reason for insertion
Explain procedure to patient
Use smallest catheter possible
Perform hand hygiene
Follow aseptic technique

Additional information may also be included, such as a fillable form that provides fields for the date and time of insertion of the catheter to be recorded, the name of the health care services provider, and the signature of the health care services provider. The above text for the instruction tag is illustrative only, and may be customized as desired by the manufacturer.

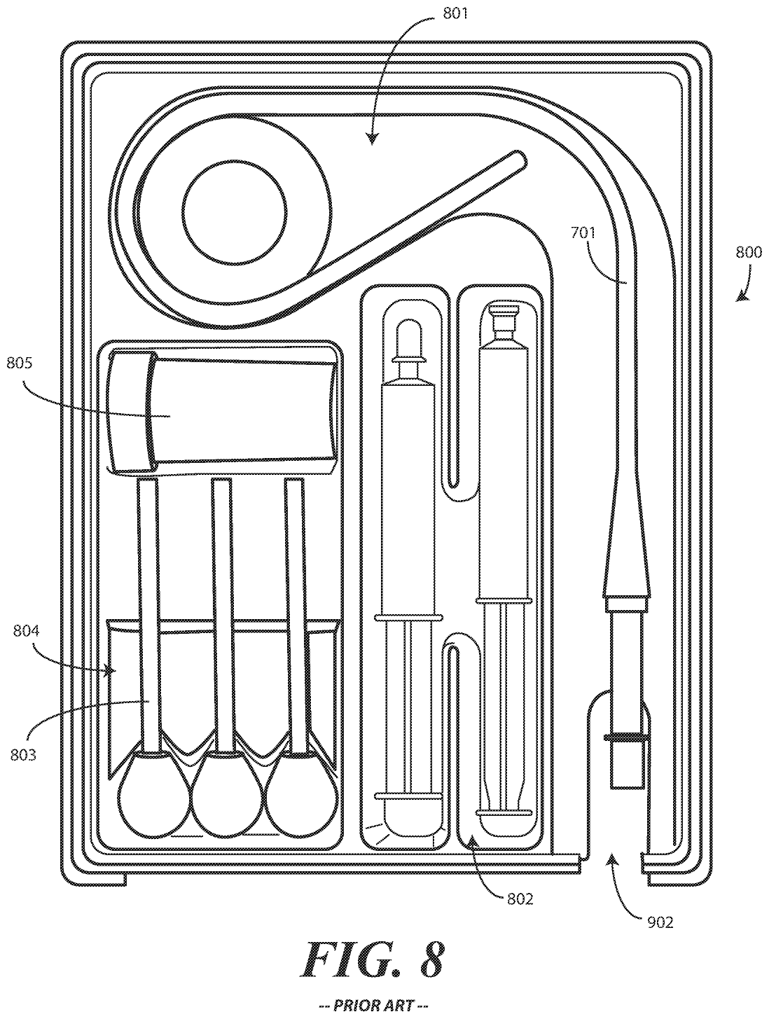

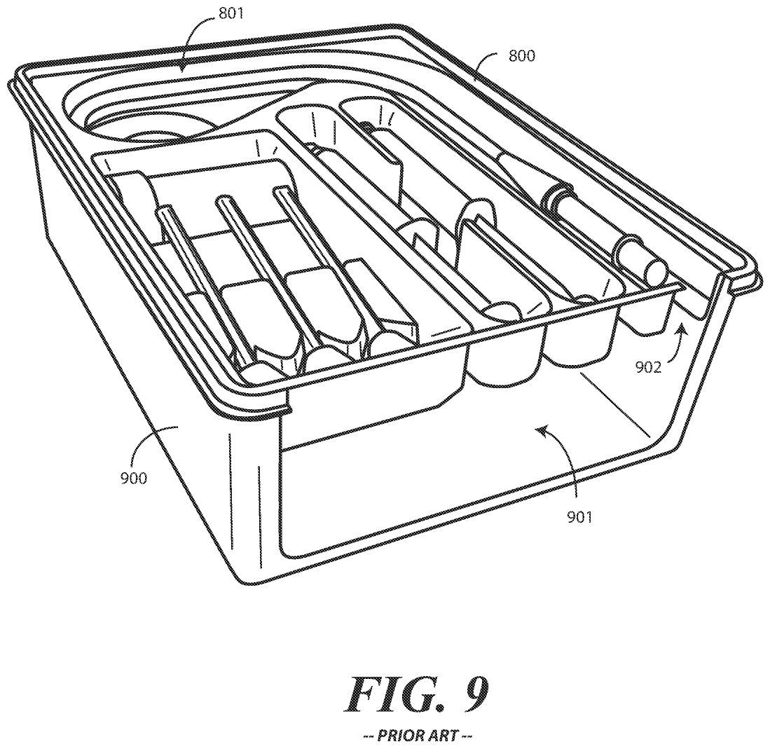

Turning now to FIG. 8, illustrated therein is another tray 800. This tray 800 is considered to be a “working tray” for a medical kit because it sits atop another tray known as a “storage tray” when the medical kit is configured in a two-layer, stacked configuration like that shown in FIG. 9.

The tray 800 houses a Foley catheter 701, while the storage tray that will be situated beneath tray 800 houses a drainage system including drainage tubing and a drainage receptacle, which could be any of a drainage bag and/or optionally including a urine meter. The tray 800 can include a number of preformed sections configured to respectively hold a number of components. For example, at least one section of the preformed sections of the tray 800 can include a catheter section 801 configured to hold the Foley catheter 701.

An example of a medical kit including both a working tray and a storage tray in a stacked configuration is shown in FIG. 9. Turning to FIG. 9, the tray 800 can be configured to nest with the storage tray 900 by suspending the tray 800 from the storage tray 900. The storage tray 900 is manufactured from cardboard, which is generally incredibly flimsy. The storage tray 900 can include an opening 901 configured as a cutout in an end of the storage tray 900. The opening 901 in the end of the storage tray 900 can be configured to facilitate access to any of the components of the kit stored in the storage tray 900 while working from tray 800.

For example, until needed, and unless it collapses due to its flimsy cardboard construction, the drainage system can be stored in the storage tray 900 while working from the upper tray, i.e., tray 800. The opening 901 also allows access to a bottom of tray 800, if needed, to lift tray 800 out of the storage tray 900 by placing a hand in the opening against the bottom of tray 800, thereby completely destroying the sterile field. That said, tray 800 can be fixed (e.g., fastened, bonded, etc.) to the storage tray 900 instead. Where this is the case, tray 800 remains fixed to the storage tray 900 throughout a catheterization procedure, which makes it all but impossible to retrieve the majority of items stored within the storage tray 900. Indeed, and wholly copying the tray of FIGS. 1-6, instructions can be included along with step-by-step catheterization instructions imprinted directly on tray 800 telling you to not separate tray 800 from the storage tray 900 unless you just want a complete and utter mess.

As noted above, the storage tray 900 is frequently nothing more than a flimsy, folded, and utterly unstable paperboard storage tray. This subpar design can be constructed from an unfolded paperboard precursor. Once the paperboard precursor is folded along the requisite fold lines, the resulting storage tray 900 includes folded-over longitudinal edges configured to engage a number of protrusions molded into tray 800, thereby forming a portion of a fastening mechanism for fastening together tray 800 and the storage tray 900.

As shown in FIGS. 8-9, tray 800 can include a cutout 902 in a corner area of the catheter section 801 formed between a bottom portion and an end portion of the catheter section 801. The cutout 902 in the comer area of the catheter section 801 can be configured to allow, for example, the drainage tubing of the drainage system to pass from the storage tray 900 to tray 800 by way of a bottom of the cutout 902 when the drainage tubing is stored in the storage tray 900. A side of the cutout 902 in the comer area of the catheter section 801 can be configured to allow the drainage tubing to be removed from the storage tray 900 without disconnecting the drainage tubing from the Foley catheter 701 when the drainage system and the Foley catheter 701 are pre-connected in the kit.

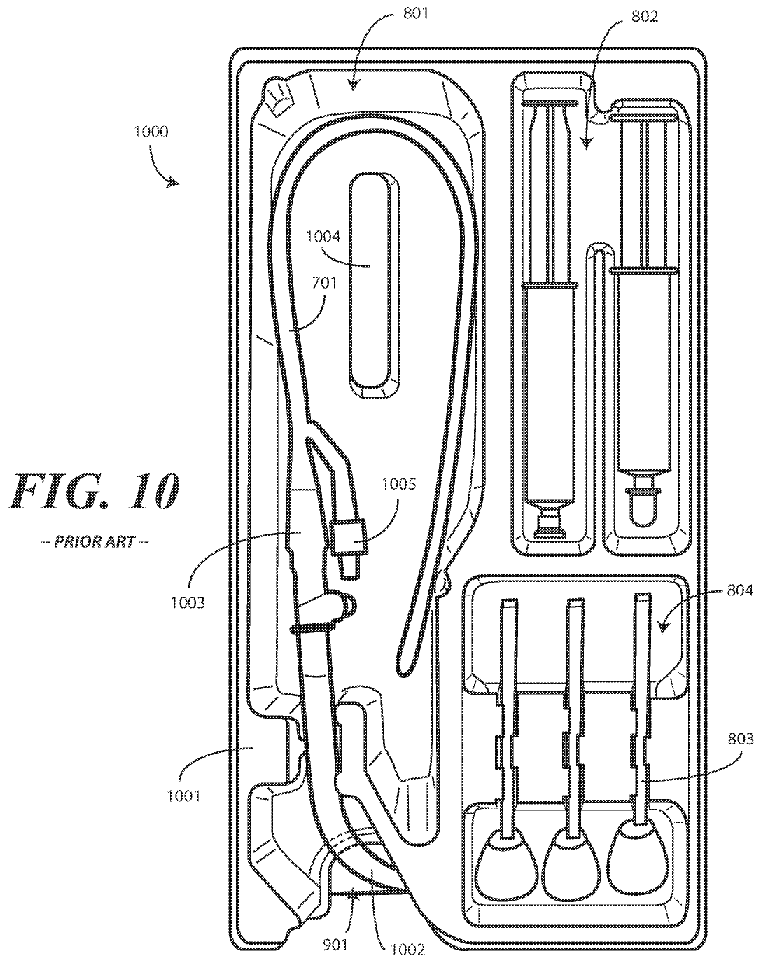

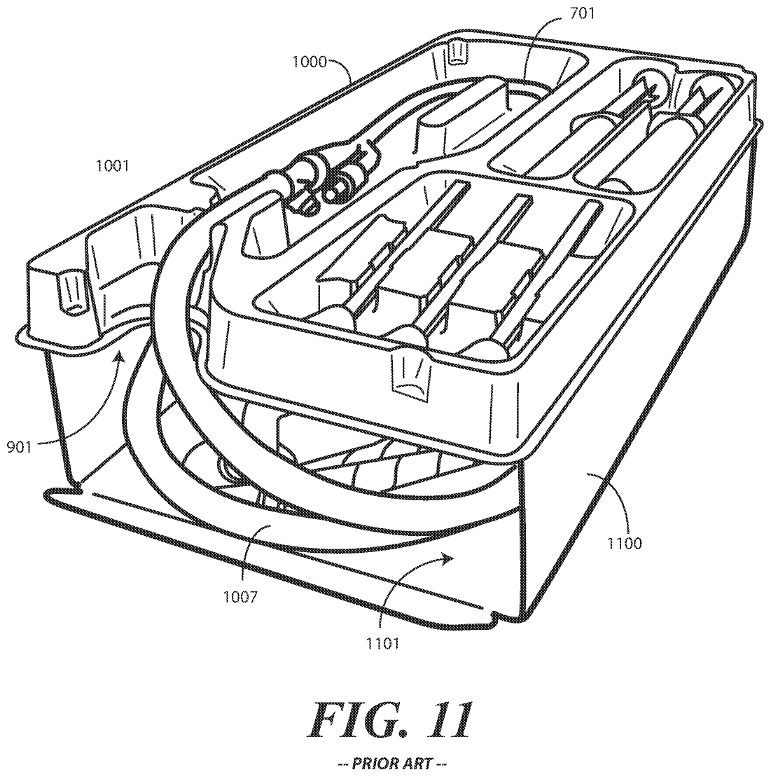

Turning now to FIG. 10, illustrated therein is another tray 1000 that is similar to tray 800 in that it can be situated atop a storage tray, as shown in FIG. 12. This tray 1000 includes a drainage-tubing constriction 1001 between the cutout 902 in the corner area of the catheter section 801. The drainage-tubing constriction 1001 can be configured to grip and hold a portion of the drainage tubing 1002 when the drainage system and the Foley catheter 701 are pre-connected.

By holding the drainage tubing 1002 in the drainage-tubing constriction 1001, an initial or as-manufactured placement of the Foley catheter 701 in the tray 800 can be maintained until the Foley catheter 701 is used for urinary catheterization. As shown, the drainage-tubing constriction 1001 can include snap-in tabs configured to really, really hold the drainage tubing 1002 in the drainage-tubing constriction 1001.

The Foley catheter 701 can be pre-connected to the drainage system. Between the Foley catheter 701 and the drainage tubing 1002, a connector 1003 can be used to connect the Foley catheter 701 and the drainage tubing 1002. The connector 1003 can include a urine-sampling port configured to facilitate urine sampling.

Referring collectively to FIGS. 8 and 10, the tray 800,1000 can include a syringe compartment 802. Syringes can be placed in the syringe compartment 802. The syringe compartment 802 can be preformed to accommodate the syringes. However, the syringe compartment 802 does not conform to every feature of the syringes. For example, the syringe compartment 802 does not include a recess for a flange of the syringe. Instead, the syringe compartment 802 has a constant width or depth commensurate with at least the flange of the syringe to accommodate the syringes. The syringe compartment 802 can have a constant width or depth commensurate with the greatest width or diameter of the syringe.

The tray 800,1000 can also house one or more swab sticks 803. These swab sticks 803 can be placed in a swab stick compartment 804. The swab stick compartment 804 can include one or more angled channels to respectively hold the one or more swab sticks 803 with their corresponding one or more absorbent heads angled downward. As shown, the one or more angled channels can include snap-in tabs configured to hold the one or more swab sticks respectively in the one or more angled channels.

The tray can also include a specimen container 805. Tray 800 includes a specimen container 805, while tray 1000 does not. Where the tray 1000 fails to include a specimen container 805, the same will have to be placed in the storage tray (900) where it will have to be dug out amongst all the other medical implements stuffed therein. Configuring the storage tray (900) to hold the specimen container instead of tray 1000 minimizes a height of tray 1000 while greatly increasing both the difficulty of retrieving the specimen container 805 as well as increasing the chances of destroying the sterile field.

The tray 1000 of FIG. 10 includes an island barrier 1004 in the catheter section 801. As explained at paragraph [0066] of WO2019/246307, instructions should be placed within the catheter section 801 expressly or implicitly suggest dispensing lubricant from one of the syringes into the catheter section 801 for lubricating the Foley catheter 701. This feature is taken from the tray of FIGS. 1-6.

The problem with this island barrier is that dispensing lubricant into the catheter section 801 will totally gunk up both the connector 1003 and the urine sampling port 1005. This is true because the lubricant will pass between the island barrier 1004 and the drainage-tubing constriction 1001.

Turning now to FIG. 11, illustrated therein is the tray 1000 of FIG. 10 positioned atop another flimsy, cardboard storage tray 1100. As shown, medical implements of all sorts are simply jammed into the storage tray 1100 with no rhyme or reason. Getting the Foley catheter 701 out of the tray 1000 without compromising the sterile field, especially when the drainage-tubing constriction 1001 has a death grip on the coiled tubing 1002, is all but impossible. Moreover, as noted above, the flimsy, cardboard storage tray 1100 is likely to collapse as well.

As before, the flimsy, cardboard storage tray 1100 includes an opening 1101 configured as a cutout in an end of the storage tray 1100. The opening 1101 in the flimsy, cardboard storage tray 1100 is on the same end of the kit formed by stacking tray 1000 atop tray 1100 as is the cutout of tray 1000. The opening 1101 in the end of the storage tray 1100 facilitates, but does not make easy, access to any of the components of the kit casually packed in the storage tray 1100 while working from tray 1000 as previously described.

Turning now to FIG. 12, illustrated therein is one explanatory kit comprising a tray 1200 and a storage tray 1202 configured in accordance with embodiments of the disclosure. The tray 1200 includes many features in common with the tray (1000) of FIG. 10. These features include a cutout 902 in a corner area of the catheter section 1201 formed between a bottom portion and an end portion of the catheter section 1201 that allows the drainage tubing 1002 of the drainage system to pass from the storage tray 1202 to tray 1200 by way of a bottom of the cutout 902 when the drainage tubing 1002 is stored in the storage tray 1200. The common features also include a Foley catheter 701 connected to the drainage system, as well as a connector 1003 situated between the Foley catheter 701 and the drainage tubing 1002 and a urine sampling port 1005 configured to facilitate urine sampling.

The tray 1200 also includes a syringe compartment 802. Syringes can be placed in the syringe compartment 802. The syringe compartment 802 is preformed to accommodate the syringes. The tray 1200 additionally houses one or more swab sticks 803, with the swab sticks 803 being placed in a swab stick compartment 804 that includes one or more angled channels to respectively hold the one or more swab sticks 803 with their corresponding one or more absorbent heads angled downward.

The tray 1200 of FIG. 12 differs from the tray (1000) in four critical respects: First, there is no drainage-tubing constriction (1001) between the cutout 902 in the corner area of the catheter section 1201 that is configured to hold a portion of the drainage tubing 1002 in the tray 1200. Second, the tray 1200 includes no island barrier (1004). Instead, it includes a big, beautiful bar, configured as a peninsular barrier 1203 that extends from an end of the tray 1200 to a distance exceeding seventy-five percent of the length of the catheter section 1201. Third, the tray 1200 includes a plurality of catheter retention devices 1204,1205,1206,1207 that are each configured to retain a different portion of the Foley catheter 701 within the catheter section 1201. Finally, rather than being manufactured from flimsy cardboard, the storage tray 1202 of FIG. 12 is a rigid tray manufactured from a thermoplastic material.

As shown in FIG. 12, rather than including the drainage-tubing constriction (1001) between the cutout 902 in the corner area of the catheter section (801) with its death grip that makes removing the Foley catheter 701 from the catheter section, the tray 1200 of FIG. 12 includes a plurality of catheter retention devices 1204,1205,1206,1207 each configured to retain a different portion of the Foley catheter 701 within the Foley catheter compartment defined by the catheter section 1201 of the tray. While four catheter retention devices 1204,1205,1206,1207 are shown in FIG. 12, it should be noted that there could be more, or fewer, than four catheter retention devices 1204,1205,1206,1207 in other embodiments.

In one or more embodiments, each catheter retention device 1204,1205,1206,1207 is configured as a retaining device defining an aperture through which a portion of the Foley catheter 701 may pass. In this illustrative embodiment, each catheter retention device 1204,1205,1206,1207 comprises a protrusion extending from a base 1209 of the tray 1200. Each protrusion defines an aperture 1208 into which the Foley catheter 701 inserts when the Foley catheter 701 is positioned within the Foley catheter compartment defined by the catheter section 1201 of the tray 1200.

Illustrating by example, in the sectional view of catheter retention device 1205, a polygonal retaining device defines an aperture 1208 through which a section of the Foley catheter 701 may pass when the Foley catheter 701 is initially loaded into the Foley catheter compartment defined by the catheter section 1201 of the tray 1200. As noted above, the Foley catheter 701 is a flexible device. Accordingly, when loading the Foley catheter 701 into the Foley catheter compartment defined by the catheter section 1201 of the tray 1200, the catheter portion passes through the aperture of each catheter retention device 1204,1205,1206,1207 so as to wrap around the peninsular barrier 1203 into the configuration shown in FIG. 12.

To remove the Foley catheter 701, rather than having to wrestle, tug, and pull on the Foley catheter 701 and/or the drainage tubing 1002 to try and free the drainage tubing 1002 from the drainage-tubing constriction (1001), which could damage the Foley catheter 701 and/or compromise the sterile field, in the embodiment of FIG. 12 one simply pulls the connector 1003 toward the cutout 902 (toward the top of the page as viewed in FIG. 12) to slide the catheter portion through the apertures of each catheter retention device 1204,1205,1206,1207. This provides a simple, quick, easy, and convenient way to release the Foley catheter 701 from the catheter section 1201 of the tray 1200.

While a retaining device defining an aperture 1208 completely encircling the portion of the Foley catheter 701 is one illustrative example of a catheter retention device, embodiments of the disclosure are not so limited. The catheter retention devices could also comprise retaining devices that partially encircle the portions of the Foley catheter 701. Alternatively, the catheter retention devices could comprise clips, clamps, clasps, braces, fasteners, catches, hooks, or other retention devices configured to retain the Foley catheter 701 within the catheter section 1201.

These catheter retention devices could be integrated into the tray 1200 as shown in FIG. 12, or could alternatively be configured as separate components that mechanically couple to the tray 1200 or a feature of the tray to retain the Foley catheter 701 within the catheter section 1201. Regardless of configuration, in one or more embodiments the tray 1200 includes one or more catheter retention devices, which can be configured as clamps, clips, or otherwise, and which hold the Foley catheter 701 better in place within the catheter section 1201 of the tray. In still other embodiments, the catheter retention devices define little arches through which the Foley catheter 701 may pass.

The idea behind the inclusion of catheter retention devices situated within the catheter section 1201 of the tray is that many nurses or other health care service providers will lift the tray 1200 off of the storage tray 1202 of the two-layer tray system. When they do, and when the drainage tubing 1002 is at least partially stowed within the storage tray 1202, the Foley catheter 701 will likely go all over the place, even when the drainage tubing 1002 is retained in the tray by a drainage-tubing constriction (1001). While the protrusions of the drainage-tubing constriction (1001) can apply a substantial grip to the drainage tubing 1002, they fail to prevent the drainage tubing 1002 from pivoting when the tray 1200 moves relative to the storage tray 1202. Advantageously, the catheter retention devices retain different portions of the Foley catheter 701 itself, rather than the drainage tubing 1002, within the Foley catheter compartment defined by the catheter section 1201 of the tray 1200. Note that while these catheter retention devices are illustratively shown in the tray of FIG. 12, they could also be applied to any of the previously described trays if those trays were modified in accordance with embodiments of the disclosure.

Accordingly, as shown in FIG. 12 a medical procedure kit comprises a first tray, namely, storage tray 1202, and a second tray, namely, tray 1200, nested atop and suspended by the first tray. Tray 1200 defines a plurality of preformed compartments such as the catheter section 1201, the syringe compartment 802, and the swab stick compartment 804 configured to receive and house a plurality of medical implements such as the Foley catheter 701, the syringes, and the swab sticks 803. The Foley catheter 701 is positioned within a Foley catheter compartment defined by the catheter section 1201, while a drainage system is at least partially stowed within an interior of the storage tray 1202. The drainage system comprises at least a portion of drainage tubing 1002, which is coiled in this illustrative embodiment, and is coupled between the Foley catheter 701 and a fluid drain bag stowed within the storage tray 1202. Tray 1200 defines a plurality of catheter retention devices 1204,1205,1206,1207 each configured to retain a different portion of the Foley catheter 701 within the Foley catheter compartment defined by the catheter section 1201 of the tray 1200.

Another distinction of the tray 1200 of FIG. 12 is the big, beautiful bar defined by the peninsular barrier 1203. As shown in FIG. 12, the peninsular barrier 1203 defines a barrier wall extending from an end of the Foley catheter compartment defined by the catheter section 1201 of the tray across a majority of the length of the Foley catheter compartment toward a distal end of the Foley catheter compartment.

In one or more embodiments, the peninsular barrier 1203 that extends from an end of the tray 1200 to a distance exceeding seventy-five percent of the length of the catheter section 1201. In this illustrative embodiment, the peninsular barrier 1203 extends across more than ninety-percent of the length of the catheter section 1201. Advantageously, this allows the peninsular barrier 1203 to situate between the urine sampling port 1005 coupled to the connector 1003 connecting the Foley catheter 701 and the drainage tubing 1002 and at least some of the distal end of the Foley catheter 701. This advantageously prevents lubrication from gunking up the urine sampling port 1005 when lubricating jelly is dispensed from one of the syringes into the catheter section 1201.

Recall from the discussion of the tray (1000) of FIG. 10 that the island barrier (1004) allows dispensed lubricant into the catheter section (801) of that tray (1000) to totally gunk up both the connector 1003 and the urine sampling port 1005 due to the fact that the lubricant will pass between the island barrier (1004) and the drainage-tubing constriction (1001). The tray 1200 of FIG. 12 advantageously prevents this from occurring by omitting the island barrier, omitting the drainage-tubing constriction, adding the one or more catheter retention devices, and extending the peninsular barrier 1203 across a majority of the Foley catheter compartment defined by the catheter section 1201. Thus, while the Foley catheter 701 comprises a connector 1003 including a urine sampling port 1005, the barrier wall defined by the peninsular barrier 1203 advantageously situates between the urine sampling port 1005 and at least some of the distal end of the catheter portion of the Foley catheter 701, thereby preventing lubricant applied to the distal end of the catheter portion of the Foley catheter 701 from gunking up the urine sampling port 1005.

Yet another distinction offered by the kit of FIG. 12 is that the storage tray 1202 is not manufactured from flimsy cardboard. Instead, the storage tray 1202 comprises a rigid tray manufactured from a thermoplastic material.

In one or more embodiments, the storage tray 1202 is thermally formed on a mold from a thermoplastic, such as styrene or polystyrene. In another embodiment, the storage tray 1200 can be injection molded from a thermoplastic material such as ABS, polycarbonate, or polycarbonate-ABS, PMMA, PVC, or other polyamide-based thermoplastics. Other suitable materials for manufacturing the storage tray 1202 will be obvious to those of ordinary skill in the art having the benefit of this disclosure. In another embodiment, the storage tray 1200 can be poured on a mold using a quick setting plastic, epoxy, or resin. Other methods of manufacture will be obvious to those of ordinary skill in the art having the benefit of this disclosure.

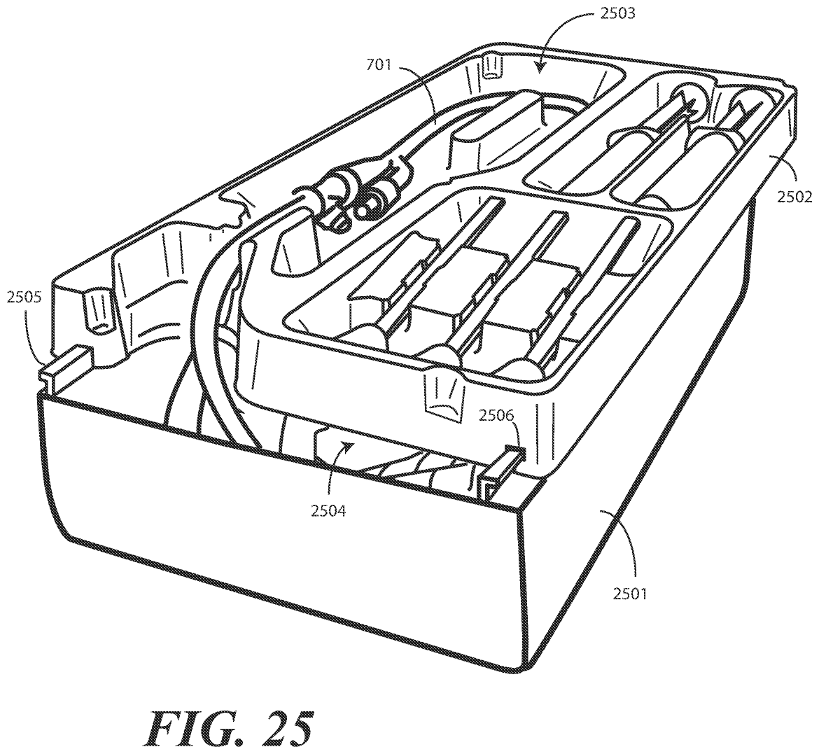

By making the storage tray 1202 from a rigid, thermoplastic material rather than flimsy cardboard, the aforementioned collapsing problem is eliminated. Additionally, as will be described below with reference to FIG. 25, features can be incorporated into the storage tray 1202 such as rails and other attachment mechanisms that allow the upper tray to slide relative to a lower tray, and so forth. Other advantages of making the storage tray 1202 rigid from a thermoplastic material will be obvious to those of ordinary skill in the art having the benefit of this disclosure.

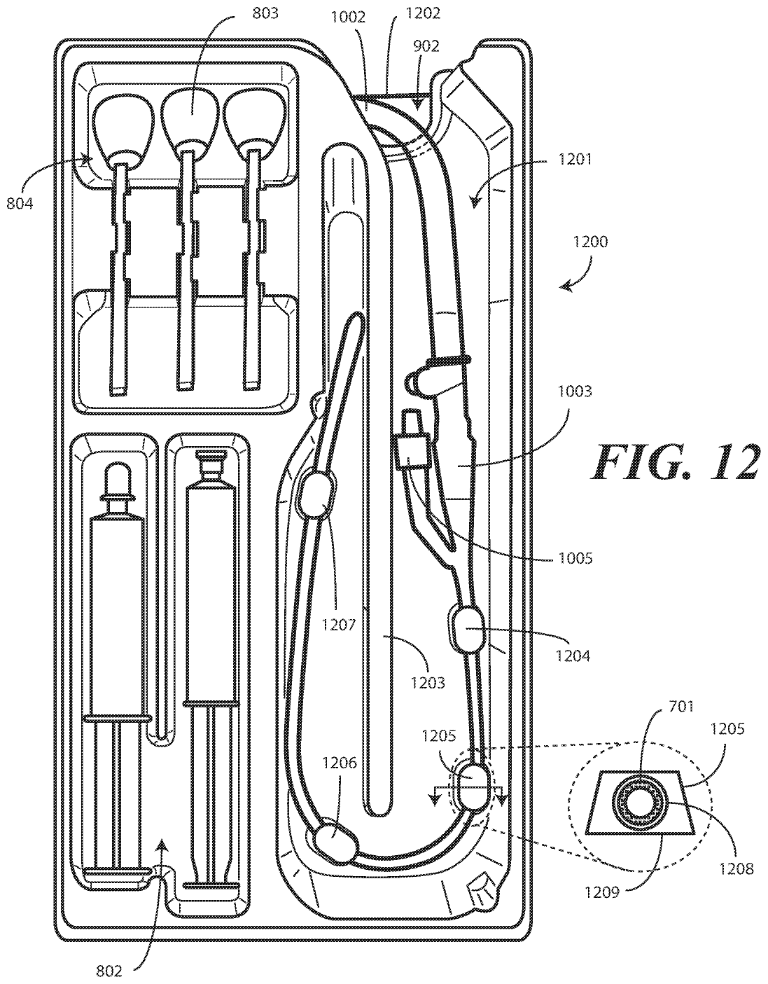

Turning now to FIG. 13, illustrated therein is the underside of the tray 1000 of FIG. 10. The tray 1000 includes a number of preformed sections configured to hold a number of components. The preformed sections of the 1000 include at least the catheter section 801, the syringe compartment 802, and the swab stick compartment 804.

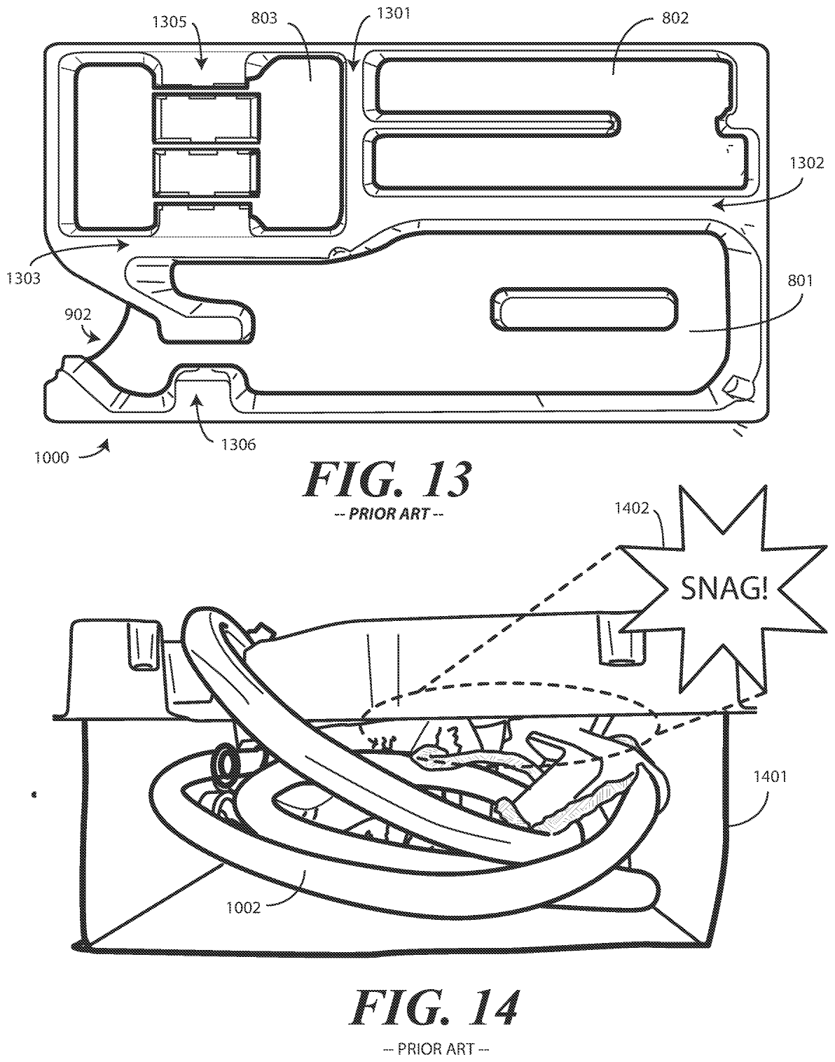

Since the tray is made with the various compartments extending from an edge of the tray 1000, including the catheter section 801, the syringe compartment 802, and the swab stick compartment 804, these each define bumps, ridges, and contours having space gaps 1301,1302,1303 therebetween. Additionally, the leading edge of the cutout 902 creates a grabbing structure extending from the upper surfaces 1305,1306 of the tray 1000. Each of these bumps, ridges, contours and grabbing structures extend downward into the storage tray when the tray 1000 is coupled to a storage tray. As shown in FIG. 14, when lots of items are packed into the flimsy cardboard storage tray 1401, they will snag 1402 along the these bumps, ridges, contours and grabbing structures extending downward into the storage tray 1401. This is especially true with the drainage tubing 1002, which is coiled and frequently ensnares the other medical implements stored within the storage tray 1401.

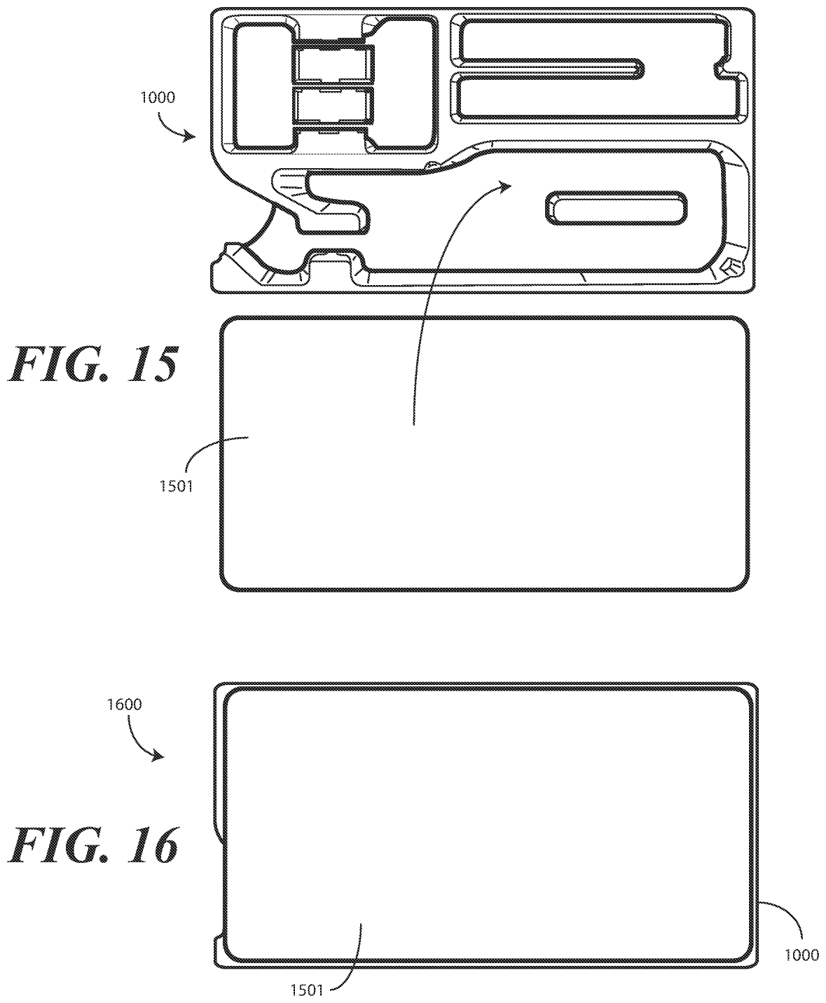

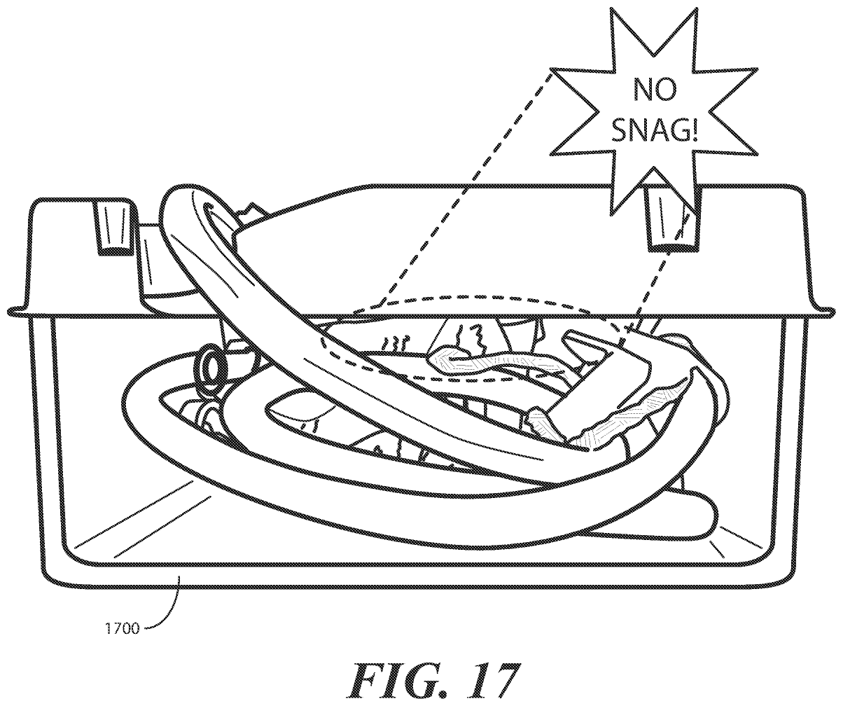

Turning now to FIG. 15, illustrated therein is a solution to this problem provided by embodiments of the disclosure. Specifically, a planar substrate 1501, which can be manufactured from plastic, cardboard, or another material, is provided to span a bottom of the bumps, ridges, contours and grabbing structures of tray 1000. The planar substrate 1501 is placed against the these bumps, ridges, contours and grabbing structures extending downward beneath the tray 1000 to form an assembly 1600 shown in FIG. 16. In one or more embodiments, the planar substrate 1501 substantially spans the bottom of the tray 1000. When this assembly 1600 is applied to a storage tray, such as the rigid, thermoplastic storage tray 1700 shown in FIG. 17, all snagging caused by the bumps, ridges, contours and grabbing structures that extend downward into the storage tray 1700 are eliminated due to the smooth surface of the planar substrate (1501) which spans not only the bumps, ridges, contours and grabbing structures of the tray 1000, but also the space gaps (1301,1302,1303) as well. Snags instantly disappear as shown thanks to the substantially planar surface provided by the planar substrate (1501).

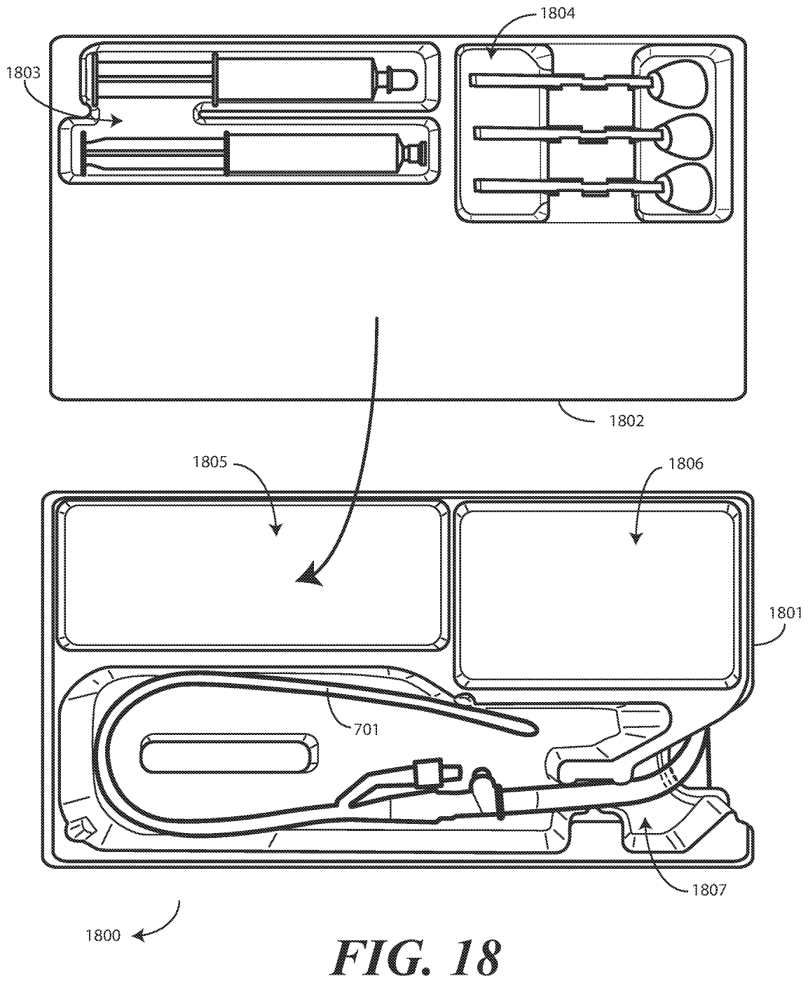

Turning now to FIG. 18, illustrated therein is a tray assembly 1800, shown in a separated view, for a stacked medical kit configured in accordance with one or more embodiments of the disclosure. The tray assembly 1800 includes a first tray 1801 and a second tray 1802 that can situate together, or can be separated apart as shown in FIG. 18. When combined with the second tray 1802 situated atop the first tray 1801, the combined first tray 1801 and the second tray 1802 serve as a working tray for a stacked medical kit.

In one or more embodiments, the second tray 1802 is configured to situate atop, but be separable from, the first tray 1801. The second tray 1802 defines a plurality of preformed compartments. In the illustrative embodiment of FIG. 18, the second tray 1802 defines a syringe compartment 1803 and a swab stick compartment 1804. As shown, one or more syringes can be positioned within the syringe compartment 1803 while one or more swab sticks can be positioned within the swab stick compartment 1804.

The first tray 1801, like the second tray 1802, also defines a plurality of preformed compartments. In this illustrative embodiment, the preformed compartments of the first tray 1801 fall into two types: compartments for receiving the preformed compartments of the second tray 1802 so that the second tray 1802 nests within the first tray 1801 and medical implement receiving compartments.

In one or more embodiments, the compartments for receiving the preformed compartments of the second tray 1802 so that the second tray 1802 nests within the first tray 1801 comprise a syringe compartment receiving compartment 1805 and a swab stick compartment receiving compartment 1806. When the second tray 1802 nests within the first tray 1801, the syringe compartment 1803 of the second tray 1802 nests within the syringe compartment receiving compartment 1805 of the first tray 1801. Similarly, the swab stick compartment 1804 of the second tray 1802 nests within the swab stick compartment receiving compartment 1806 of the first tray 1801.

In one or more embodiments, the medical implement compartments include a Foley catheter compartment 1807 configured to receive a Foley catheter 701. As shown in FIG. 18, the Foley catheter 701 is positioned within the Foley catheter compartment 1807, which is one of a plurality of compartments defined by the first tray 1801. Thus, the first tray 1801 has a Foley catheter positioned within a Foley catheter compartment 1807 of the plurality of compartments of the first tray 1801. Effectively, the assembly 1800 of FIG. 18 divides the “working tray” of FIG. 10 in two. As shown in FIG. 18, the swab sticks and syringes are one tray, while the Foley catheter 701 is in a second tray.

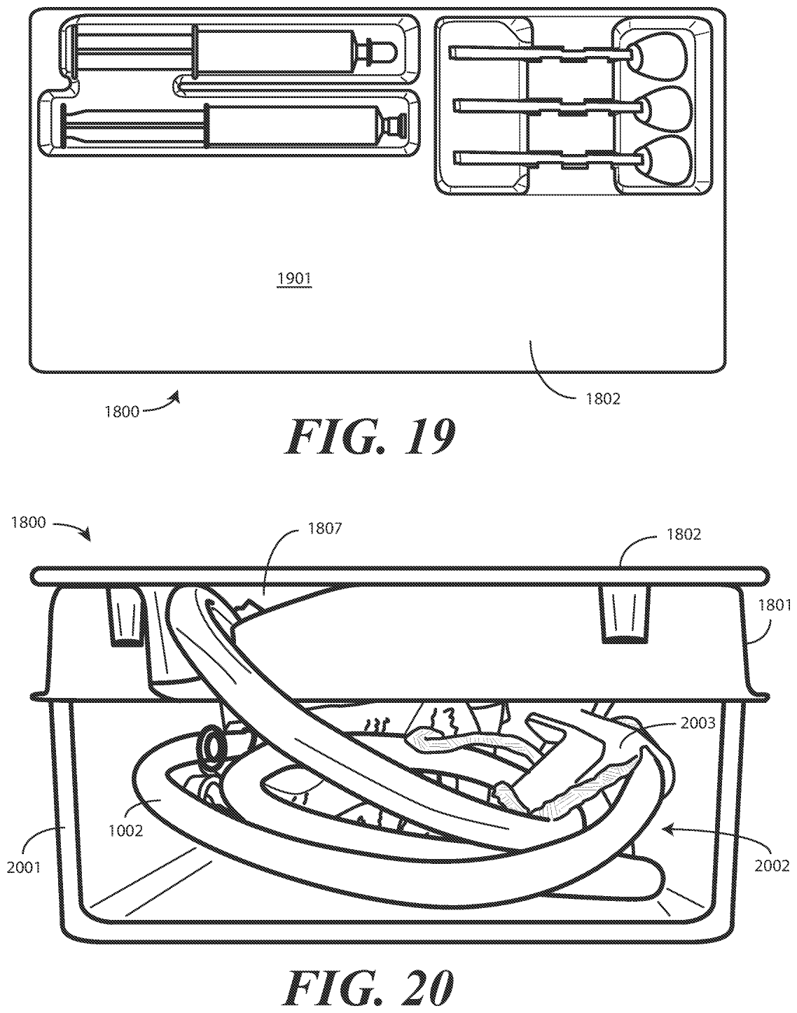

Turning now to FIG. 19, the second tray 1802 has been situated atop, and nested in, the first tray (1801). This assembly 1800 allows access to the syringes and swab sticks, but conceals the Foley catheter (701) beneath a planar portion 1901 of the second tray.

Turning now to FIG. 20, illustrated therein is a medical kit using the assembly 1800 of FIGS. 18-19. The medical kit comprises a first tray, i.e., storage tray 2001 manufactured from a rigid thermoplastic material, with a second tray, i.e., tray 1802, nested atop, and suspended by, the first tray. As previously described, the second tray defines a plurality of preformed compartments configured to receive and house a plurality of medical implements, including the Foley catheter (701), the connector coupled to the Foley catheter (701), the urine sampling port connected to the connector, and a portion of the drainage tubing 1002. As also described above, the Foley catheter (701) is positioned within a Foley catheter compartment 1807 of the plurality of preformed compartments of the second tray.

A drainage system 2002 is stowed within an interior of the first tray. The drainage system comprises at least a portion of the coiled tubing 1002 coupled between the Foley catheter (701) and a fluid drain bag 2003.

A third tray, here tray 1802, situates atop, but is separable from the second tray. The third tray defines another plurality of preformed compartments, namely, the syringe compartment (1803) and the swab stick compartment (1804). This other plurality of preformed compartments situates within a subset of the plurality of preformed compartments of the second tray, namely, the syringe compartment receiving compartment (1805) and a swab stick compartment receiving compartment (1806), when the third tray situates atop the second tray as shown in FIG. 20.

This assembly advantageously makes it easier to get the medical implements situated in the first tray 2001 out without having the issues with the Foley catheter (701) moving around in the second tray due to its confinement by the third tray. Moreover, the syringes and swab sticks can be accessed without disturbing or contaminating the Foley catheter (701).

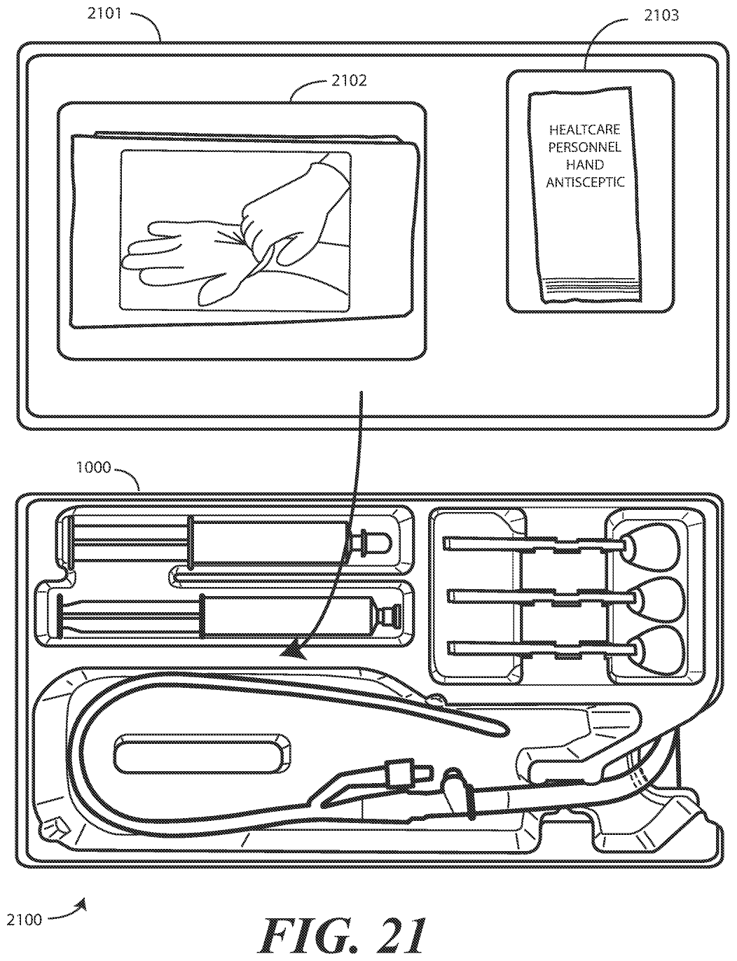

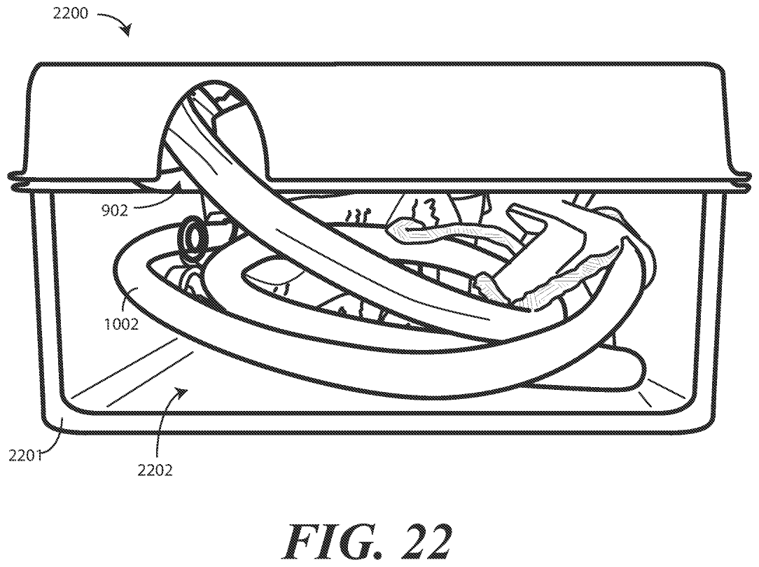

Turning now to FIG. 21, illustrated therein is yet another assembly 2100 configured in accordance with one or more embodiments of the disclosure. FIG. 21 adds yet another tray 2101 that can situate atop either the tray 1000 of FIG. 10, as shown in the figure, or atop the assembly (1800) of FIGS. 18-20. In the latter scenario, the tray 2101 comprises a fourth tray that situates atop, but is separable from, the third tray, which was tray (1802) in FIG. 20. In the illustrative embodiment of FIG. 21, the tray 2101 situates atop tray 1000. When tray 1000 is placed atop a storage tray, as described below with reference to FIG. 22, tray 2101 comprises a third tray that situates atop, but is separable from, a second tray in a stacked medical kit.

In one or more embodiments, tray 2101 defines a glove receiving compartment 2102. As shown in FIG. 21, one or more gloves may be stowed within the glove receiving compartment 2102. Similarly, the tray 2101 also defines a hand sanitizer container receiving compartment 2103. A container of hand sanitizer may be stowed within the hand sanitizer receiving compartment as shown in FIG. 21. Drapes and other medical implements can additionally be stowed within contours and recesses of tray 2101. The assembly 2100 of FIG. 21 advantageously places combinations of gloves, hand sanitizer, and/or drapes in a separate tray so that a medical practitioner need not reach into the sterile field to retrieve them.

The assembled kit 2200 is shown in FIG. 22. Note that in FIG. 22, the opening 2202 defined by the storage tray 2201 and the cutout 902 in the second tray that allows passage of the drainage tubing 1002 from the storage tray 2201 to the second tray 2201 are on the same side of the kit 2200. This was also the case for the kits shown in FIGS. 9, 11, 14, 17, and 20.

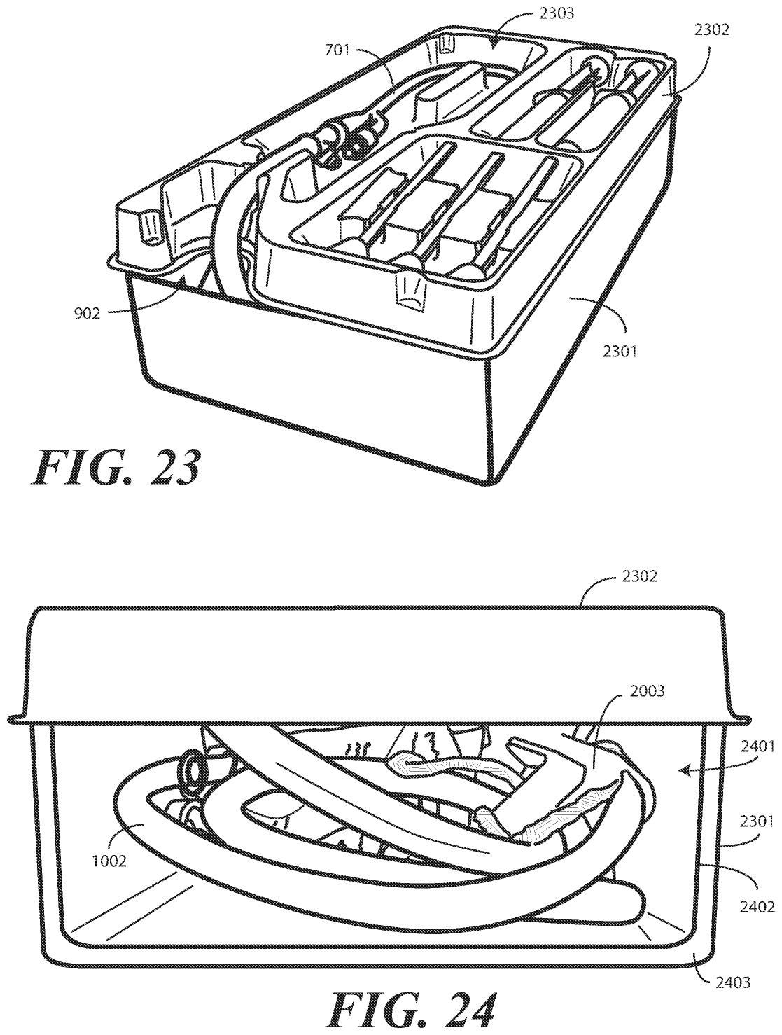

Embodiments of the disclosure contemplate that this configuration can be problematic in that a medical services provider will have to lift the working tray and rotate the same 180-degrees during a catheterization procedure if they place the opening 2202 of the storage tray 2201 toward the patient so as to allow the drainage tubing 1002 to slide out toward the patient. Said differently, the medical services provider will have to rotate the top tray 180-degrees when positioning if they put the top tray towards the patient, i.e., to the left of the bottom tray if patient is to the left. Embodiments of the disclosure also contemplate that it would be more advantageous to rotate the upper tray, as this reduces the chances for spillage or sterile field contamination. Turning now to FIGS. 23-24, illustrated therein is one such embodiment doing just this.

As shown in FIGS. 23-24, a medical procedure kit includes a first tray 2301 and a second tray 2302 nested atop, and suspended by, the first tray 2301. As before, the second tray 2302 defines a plurality of preformed compartments configured to receive and house a plurality of medical implements, many of which are shown in FIG. 23. Illustrating by example, a Foley catheter 701 is positioned within a Foley catheter compartment 2303 of the plurality of preformed compartments of the second tray 2302. Additionally, a drainage system 2401 is stowed within an interior of the first tray 2301. As before, the drainage system comprises at least a portion of a drainage tubing (which is coiled in FIG. 24) coupled between the Foley catheter 701 and a fluid drain bag 2003.

As shown in FIGS. 23-24, the first tray 2301 defines a cutout 2402 in a first end 2403 of the first tray 2301 allowing access to the components (such as the drainage system 2401) stowed within the first tray 2301. The second tray 2302 defines another cutout 902 in a corner area of the Foley catheter compartment 2303. At least some of the coiled tubing 1002 passes from the first tray 2301 into the Foley catheter compartment 2303 through this other cutout 902. As can be seen by comparing FIGS. 23 and 24, in this illustrative embodiment the cutout 2402 of the first tray 2301 and the cutout 902 of the second tray 2302 are situated at opposite ends of the medical procedure kit. This embodiment places a bottom tray having an open end and a closed end such that the closed end bounds an opening for the drainage tubing 1002, while the open end is placed on the other side. The embodiment of FIGS. 23-24 advantageously positions the top tray rotated from the start, thereby reducing the chances of a spill occurring.

Turning now to FIG. 25, illustrated therein is still another medical kit configured in accordance with one or more embodiments of the disclosure. In one or more embodiments, the medical kit includes a first tray 2501 and a second tray 2502 situated atop the first tray 2501. The second tray 2502 defines a plurality of preformed compartments configured to receive and house a plurality of medical implements. A Foley catheter 701 is positioned within a Foley catheter compartment 2503 of the plurality of preformed compartments of the second tray 2502. A drainage system 2504 is stowed within an interior of the first tray 2501. The drainage system 2504 comprises at least a portion of a coiled tubing coupled between the Foley catheter 701 and a fluid drain bag.

In one or more embodiments, the first tray 2501 slides relative to the second tray 2502. Illustrating by example, the first tray 2501 can slide out out from the second tray 2502. IN the illustrative embodiment of FIG. 25, the first tray 2501 defines a rail 2505, while the second tray 2502 defines a receiver 2506. In other embodiments, the first tray 2501 could define the receiver 2506, while the second tray defines the rail 2505. In one or more embodiments, the rail 2505 and receiver 2506 are configured to interlock, thereby allowing the second tray 2502 to slide laterally atop the first tray 2501.

Accordingly, FIG. 25 illustrates a two-layer system where the upper tray slides, optionally on rails, along the top of the bottom tray. In alternate embodiments the rail and receiver could be replaced by a channel connection so that the top tray slides out from the bottom tray. This makes the Foley catheter 701 less likely to move.

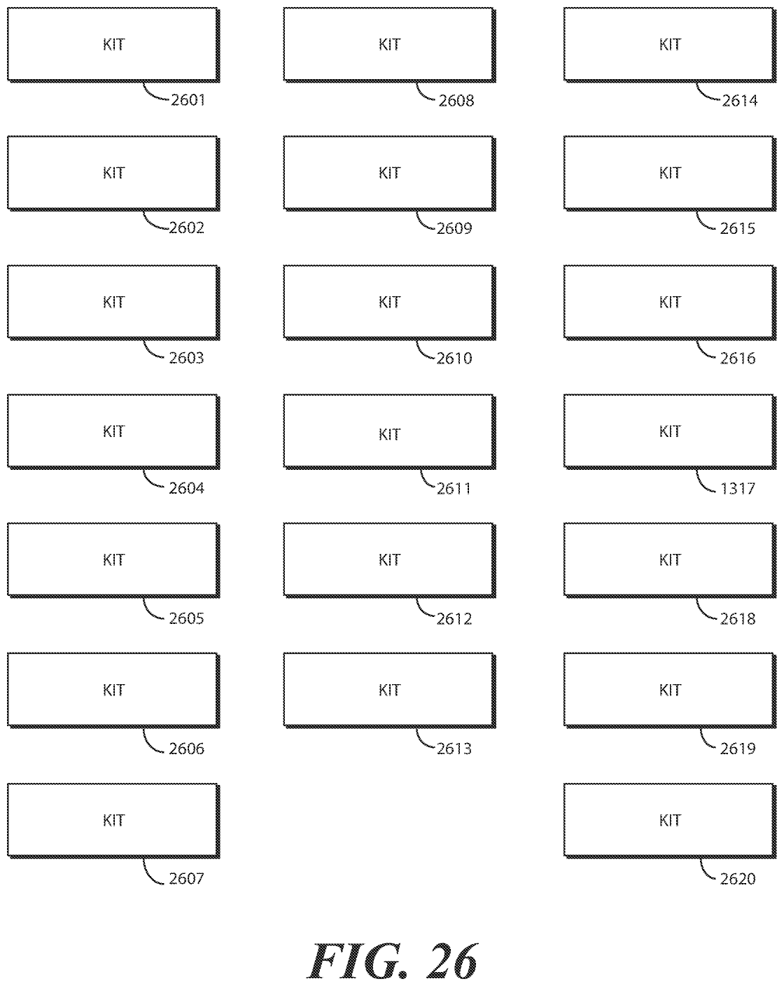

Turning now to FIG. 26, illustrated therein are various embodiments of the disclosure. The embodiments of FIG. 26 are shown as labeled boxes in FIG. 26 due to the fact that the individual components of these embodiments have been illustrated in detail in FIGS. 1-25, which precede FIG. 26. Accordingly, since these items have previously been illustrated and described, their repeated illustration is no longer essential for a proper understanding of these embodiments. Thus, the embodiments are shown as labeled boxes.

At 2601, a medical procedure kit comprises a first tray. At 2601, the medical procedure kit comprises a second tray nested atop, and suspended by, the first tray. At 2601, the second tray defines a plurality of preformed compartments configured to receive and house a plurality of medical implements.

At 2601, the medical procedure kit comprises a Foley catheter positioned within a Foley catheter compartment of the plurality of preformed compartments of the second tray. At 2601, the medical procedure kit comprises a drainage system stowed within an interior of the first tray. At 2601, the drainage system comprises at least a portion of a coiled tubing coupled between the Foley catheter and a fluid drain bag. At 2601, the medical procedure kit comprises a third tray situated atop, and separable from, the second tray.

At 2602, the third tray of 2601 defines another plurality of preformed compartments configured to situate within a subset of the plurality of preformed compartments of the second tray when the third tray situates atop the second tray. At 2603, the first tray of 2602 comprises a rigid tray manufactured from a thermoplastic material.

At 2604, the first tray of 2602 defines a cutout in an end of the first tray allowing access to components stowed within the first tray. At 2604, the second tray defines another cutout in a corner area of the Foley catheter compartment. At 2604, at least some of the coiled tubing passes from the first tray into the Foley catheter compartment through the other cutout. AT 2604, the cutout and the other cutout are on opposite ends of the medical procedure kit.

At 2605, the other plurality of preformed compartments of 2602 comprises a swab stick compartment. At 2605, the medical procedure kit further comprises one or more pre-saturated swab sticks stowed within the swab stick compartment.

At 2606, the other plurality of preformed compartments of 2605 comprises a syringe receiving compartment. At 2606, the medical procedure kit further comprises one or more syringes stowed within the syringe receiving compartment.

At 2607, the medical procedure kit of 2601 further comprises a fourth tray situated atop, and separable from, the third tray. At 2608, the fourth tray of 2607 defines a glove receiving compartment. At 2608, the medical procedure kit further comprises one or more gloves stowed within the glove receiving compartment. At 2609, the fourth tray of 2607 defines a hand sanitizer container receiving compartment. At 2609, the medical procedure kit further comprises a container of hand sanitizer stowed within the hand sanitizer receiving compartment.

At 2610, the first tray of 2601 defines one of a rail or a receiver. At 2610, the second tray defines another of a rail or a receiver configured to interlock with the one of a rail or a receiver, thereby allowing the second tray to slide laterally atop the first tray.

At 2611, a medical procedure kit comprises a first tray. At 2611, the medical procedure kit comprises a second tray nested atop, and suspended by, the first tray. At 2611, the second tray defines a plurality of preformed compartments configured to receive and house a plurality of medical implements.

At 2611, the medical procedure kit comprises a Foley catheter positioned within a Foley catheter compartment of the plurality of preformed compartments of the second tray. At 2611, the medical procedure kit comprises a drainage system stowed within an interior of the first tray. At 2611, the drainage system comprises at least a portion of a coiled tubing coupled between the Foley catheter and a fluid drain bag. At 2611, the second tray defines a plurality of catheter retention devices each configured to retain a different portion of the Foley catheter within the Foley catheter compartment.

At 2612, each catheter retention device of 2611 comprises a protrusion from a base of the second tray and defining an aperture into which the Foley catheter inserts when the Foley catheter is positioned within the Foley catheter compartment. At 2613, the Foley catheter compartment of 2612 defines a barrier wall extending from an end of the Foley catheter compartment across a majority of a length of the Foley catheter compartment toward a distal end of the Foley catheter compartment.

At 2614, the medical procedure kit of 2613 further comprises a connector configured to connect the Foley catheter to the coiled tubing that includes a urine sampling port. At 2614, the barrier wall is situated between the urine sampling port and at least some of the Foley catheter.

At 2615, the first tray of 2611 defines a cutout in an end of the first tray allowing access to components stowed within the first tray. At 2615, the second tray defines another cutout in a corner area of the Foley catheter compartment. At 2615, at least some of the coiled tubing passes from the first tray into the Foley catheter compartment through the other cutout. At 2615, the cutout and the other cutout are on opposite ends of the medical procedure kit.

At 2616, the medical procedure kit of 2611 further comprises a third tray situated atop, and separable from, the second tray. At 2611, the third tray defines another plurality of preformed compartments configured to situate within a subset of the plurality of preformed compartments of the second tray when the third tray situates atop the second tray.

At 2617, the other plurality of preformed compartments of 2616 comprises a swab stick compartment and a syringe receiving compartment. At 2617, the medical procedure kit further comprises one or more swab sticks stowed within the swab stick compartment. At 2617, the medical procedure kit further comprises one or more syringes stowed within the syringe receiving compartment. At 2618, the first tray of 2611 defines one of a rail or a receiver and the second tray defines another of a rail or a receiver configured to interlock with the one of a rail or a receiver, thereby allowing the second tray to slide laterally atop the first tray.

At 2619, a medical procedure kit comprises a first tray. At 2619, the medical procedure kit comprises a second tray nested atop, and suspended by, the first tray. At 2619, the second tray defines a plurality of preformed compartments configured to receive and house a plurality of medical implements.

At 2619, the medical procedure kit comprises a Foley catheter positioned within a Foley catheter compartment of the plurality of preformed compartments of the second tray. At 2619, the medical procedure kit comprises a drainage system stowed within an interior of the first tray. At 2619, the drainage system comprises at least a portion of a coiled tubing coupled between the Foley catheter and a fluid drain bag.

At 2619, the first tray defines a cutout in an end of the first tray allowing access to components stowed within the first tray. At 2619, the second tray defines another cutout in a corner area of the Foley catheter compartment. At 2619, at least some of the coiled tubing passes from the first tray into the Foley catheter compartment through the other cutout. At 2619, the cutout and the other cutout are on opposite ends of the medical procedure kit.

At 2620, the medical procedure kit of 2619 further comprises a planar substrate situated between a bottom of the second tray and the drainage system. At 2620, the planar substrate substantially spans the bottom of the second tray.

The following applications are incorporated herein by reference for all purposes: Ser. No. 14/793,455, filed Jul. 7, 2015; Ser. No. 13/860,902, filed Apr. 11, 2013; Ser. No. 13/155,026, filed Jun. 7, 2011; Ser. No. 61/352,140, filed Jun. 7, 2010; Ser. No. 61/352,155, filed Jun. 7, 2010; Ser. No. 61/428,944, filed Dec. 31, 2010; Ser. No. 61/437,796, filed Jan. 31, 2011; Ser. No. 12/495,148, filed Jun. 30, 2009; Ser. No. 12/647,515, filed Dec. 27, 2009; Ser. No. 13/153,265, filed Jun. 3, 2011; Ser. No. 15/977,944, filed May 11, 2018; PCT/US2019/038051, filed Jun. 18, 2019; and Ser. No. 13/153,300, filed Jun. 3, 2011.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims.

For example, the swab sticks may be saturated with CHG rather than PVP. Additionally, other catheters could be substituted for the Foley catheter in the various embodiments, including a 3-way Foley catheter, a Coude tip with temp sensing Foley catheter, or other type of catheter.

Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

1. A medical procedure kit, comprising:
a first tray;
a second tray nested atop, and suspended by, the first tray, the second tray defining a plurality of preformed compartments configured to receive and house a plurality of medical implements;
a Foley catheter positioned within a Foley catheter compartment of the plurality of preformed compartments of the second tray;
a drainage system stowed within an interior of the first tray, the drainage system comprising at least a portion of a coiled tubing coupled between the Foley catheter and a fluid drain bag; and
a third tray situated atop, and separable from, the second tray.

2. The medical procedure kit of claim 1, the third tray defining another plurality of preformed compartments configured to situate within a subset of the plurality of preformed compartments of the second tray when the third tray situates atop the second tray.

3. The medical procedure kit of claim 2, wherein the first tray comprises a rigid tray manufactured from a thermoplastic material.

4. The medical procedure kit of claim 2, wherein:
the first tray defines a cutout in an end of the first tray allowing access to components stowed within the first tray;
the second tray defines another cutout in a corner area of the Foley catheter compartment;
at least some of the coiled tubing passes from the first tray into the Foley catheter compartment through the another cutout; and
the cutout and the another cutout are on opposite ends of the medical procedure kit.

5. The medical procedure kit of claim 2, wherein the another plurality of preformed compartments comprises a swab stick compartment, further comprising one or more pre-saturated swab sticks stowed within the swab stick compartment.

6. The medical procedure kit of claim 5, wherein the another plurality of preformed compartments comprises a syringe receiving compartment, further comprising one or more syringes stowed within the syringe receiving compartment.

7. The medical procedure kit of claim 1, further comprising a fourth tray situated atop, and separable from, the third tray.

8. The medical procedure kit of claim 7, the fourth tray defining a glove receiving compartment, further comprising one or more gloves stowed within the glove receiving compartment.

9. The medical procedure kit of claim 7, the fourth tray defining a hand sanitizer container receiving compartment, further comprising a container of hand sanitizer stowed within the hand sanitizer receiving compartment.

10. The medical procedure kit of claim 1, wherein the first tray defines one of a rail or a receiver and the second tray defines another of a rail or a receiver configured to interlock with the one of a rail or a receiver, thereby allowing the second tray to slide laterally atop the first tray.

11. A medical procedure kit, comprising:
a first tray;
a second tray nested atop, and suspended by, the first tray, the second tray defining a plurality of preformed compartments configured to receive and house a plurality of medical implements;
a Foley catheter positioned within a Foley catheter compartment of the plurality of preformed compartments of the second tray; and
a drainage system stowed within an interior of the first tray, the drainage system comprising at least a portion of a coiled tubing coupled between the Foley catheter and a fluid drain bag;
wherein the second tray defines a plurality of catheter retention devices each configured to retain a different portion of the Foley catheter within the Foley catheter compartment.

12. The medical procedure kit of claim 11, wherein each catheter retention device comprises a protrusion from a base of the second tray and defining an aperture into which the Foley catheter inserts when the Foley catheter is positioned within the Foley catheter compartment.

13. The medical procedure kit of claim 12, wherein the Foley catheter compartment defines a barrier wall extending from an end of the Foley catheter compartment across a majority of a length of the Foley catheter compartment toward a distal end of the Foley catheter compartment.

14. The medical procedure kit of claim 13, further comprising a connector configured to connect the Foley catheter to the coiled tubing that includes a urine sampling port, wherein the barrier wall is situated between the urine sampling port and at least some of the Foley catheter.

15. The medical procedure kit of claim 11, wherein:
the first tray defines a cutout in an end of the first tray allowing access to components stowed within the first tray;
the second tray defines another cutout in a corner area of the Foley catheter compartment;
at least some of the coiled tubing passes from the first tray into the Foley catheter compartment through the another cutout; and
the cutout and the another cutout are on opposite ends of the medical procedure kit.

16. The medical procedure kit of claim 11, further comprising a third tray situated atop, and separable from, the second tray, the third tray defining another plurality of preformed compartments configured to situate within a subset of the plurality of preformed compartments of the second tray when the third tray situates atop the second tray.

17. The medical procedure kit of claim 16, wherein the another plurality of preformed compartments comprises a swab stick compartment and a syringe receiving compartment, further comprising:
one or more swab sticks stowed within the swab stick compartment; and
one or more syringes stowed within the syringe receiving compartment.

18. The medical procedure kit of claim 11, wherein the first tray defines one of a rail or a receiver and the second tray defines another of a rail or a receiver configured to interlock with the one of a rail or a receiver, thereby allowing the second tray to slide laterally atop the first tray.

19. A medical procedure kit, comprising:
a first tray;
a second tray nested atop, and suspended by, the first tray, the second tray defining a plurality of preformed compartments configured to receive and house a plurality of medical implements; and
a Foley catheter positioned within a Foley catheter compartment of the plurality of preformed compartments of the second tray; and
a drainage system stowed within an interior of the first tray, the drainage system comprising at least a portion of a coiled tubing coupled between the Foley catheter and a fluid drain bag;
wherein:
the first tray defines a cutout in an end of the first tray allowing access to components stowed within the first tray;
the second tray defines another cutout in a corner area of the Foley catheter compartment;
at least some of the coiled tubing passes from the first tray into the Foley catheter compartment through the another cutout; and
the cutout and the another cutout are on opposite ends of the medical procedure kit.

20. The medical procedure kit of claim 19, further comprising a planar substrate situated between a bottom of the second tray and the drainage system, wherein the planar substrate substantially spans the bottom of the second tray.
